(19) United States
(12) Patent Application Publication
(10) Pub. No.: US 2026/0158234 A1
(43) Pub. Date: Jun. 11, 2026

(54) MOBILE THERAPEUTIC DEVICE FOR HORSEBACK SENSORY THERAPY

(71) Applicants: William Dennert, Box Elder, SD (US); Tammy Dennert, Box Elder, SD (US)

(72) Inventors: William Dennert, Box Elder, SD (US); Tammy Dennert, Box Elder, SD (US)

(21) Appl. No.: 19/321,558

(22) Filed: Sep. 8, 2025

Related U.S. Application Data
(60) Provisional application No. 63/730,539, filed on Dec. 11, 2024.

(51) Int. Cl. A61M 21/02; A61H 1/00; A61M 21/00

(52) U.S. Cl. CPC A61M 21/02; A61H 1/005; A61H 2201/0157; A61H 2201/1215; A61H 2201/1418; A61H 2201/1654; A61H 2201/1673; A61H 2201/1676; A61H 2201/5005; A61H 2203/0437; A61M 2021/0022; A61M 2205/3365; A61M 2205/59; A61M 2205/8206

(57) ABSTRACT

A mobile therapeutic mechanical horse for horseback sensory therapy includes a first rotatable platform section, a second rotatable platform section, and a mechanical horse body including two curved sheets each having a body hinge attached to a top horizontal member, two rear legs and two front legs each hinged on a top end to one of the two curved sheets, wherein each of the two rear legs and two front legs have a bottom linkage pivotally secured to the first rotatable platform section or the second rotatable platform section. A motion assembly includes a first cam for vertical movement of the first rotatable platform section, and a second cam for vertical movement of the second rotatable platform section. A control panel includes a speed rheostat for controlling speed of the mobile therapeutic mechanical horse and a motion rheostat for controlling frequency of vertical stroke of the curved sheets.

### CROSS-REFERENCE TO RELATED APPLICATIONS

This application is a Utility Patent application claiming priority to U.S. Provisional Patent Application Ser. No. 63/730,539, filed on Dec. 11, 2024, which is incorporated by reference herein in its entirety.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The present disclosure relates to therapeutic devices for sensory therapy, and more particularly to a mobile mechanical horse that simulates equine movement to provide horseback sensory therapy in a controlled and safe environment.

### 2) Description of Related Art

The rhythmic motion and physical engagement involved in riding a horse can provide valuable sensory input and motor skill development for individuals with various physical, cognitive, or developmental challenges. However, access to live horses and suitable riding facilities can be limited due to factors such as cost, location, and safety concerns.

Horseback sensory therapy, also known as equine-assisted therapy, has been recognized as an effective treatment modality for individuals with various mental health conditions and neurological disorders. This therapeutic approach leverages the natural movement patterns and rhythmic motion of horses to provide sensory input that can help improve balance, coordination, muscle strength, and emotional well-being in patients.

Traditional horseback sensory therapy involves mounting and riding live horses under the supervision of trained therapists. The three-dimensional movement of a horse's gait provides proprioceptive and vestibular stimulation that can be beneficial for individuals with conditions such as cerebral palsy, autism spectrum disorders, post-traumatic stress disorder, and various developmental disabilities. The rhythmic motion of the horse's movement can help stimulate neural pathways and promote relaxation responses in patients.

However, traditional equine-assisted therapy faces several limitations that restrict its accessibility and application. Working with live animals introduces inherent safety risks and unpredictability factors that may not be suitable for all patients. Horses can be startled, may exhibit unexpected behaviors, or may not maintain consistent movement patterns throughout therapy sessions. Additionally, traditional horseback therapy typically has weight restrictions that limit participation to individuals under certain weight thresholds, excluding those who might otherwise benefit from this form of treatment.

The availability of suitable therapy horses and qualified facilities can also limit access to equine-assisted therapy programs. Maintaining live horses requires substantial resources, specialized facilities, and trained personnel, which can make such programs expensive and geographically limited. Weather conditions and seasonal factors can further restrict the availability of outdoor equine therapy sessions.

Furthermore, some individuals may have allergies to horses or may experience anxiety or fear when working with large animals, preventing them from participating in traditional horseback sensory therapy despite potential therapeutic benefits. The variability in horse temperament and movement patterns can also make it challenging to provide consistent therapeutic experiences across multiple sessions.

These limitations have created a need for alternative approaches that can provide the therapeutic benefits of horseback sensory therapy while addressing the accessibility, safety, and consistency challenges associated with live animal therapy. Mechanical alternatives that can simulate the beneficial movement patterns of horses while providing a controlled and predictable therapeutic environment represent a potential solution to expand access to this form of sensory therapy.

### BRIEF SUMMARY OF THE INVENTION

The instant invention in one form is directed to a mobile therapeutic mechanical horse. The mobile therapeutic mechanical horse includes a 12-volt actuated steering assembly enabling precise left and right directional control to maintain motion in a circle pattern. The mobile therapeutic mechanical horse also includes a 12-volt variable speed drive allowing forward and backward movement, with the ability to adjust speeds as needed for patient sensory therapy sessions. Additionally, the mobile therapeutic mechanical horse includes a 12-volt variable speed motion control to regulate up-and-down hip movement characteristic replicated from a horse.

The mobile therapeutic mechanical horse further includes a horizontal frame having a length and a wheel and rubber tire along each side of the frame length. A vertical frame includes an upper member extending the length of the horizontal frame. An elevated platform includes a pair of flat metal platforms hinged along adjacent edges of the pair of flat metal platforms. A pair of curved seat members are hinged along the vertical frame upper member. An elongated leg member is attached to each of the curved seat members, with each elongated leg member including one upright member end pivotally attached to each of the pair of flat metal platforms and an opposite upright member end pivotally attached to each of the pair of flat metal platforms.

The mobile therapeutic mechanical horse also includes a cam and cam shaft consecutively cycling each of the pair of metal platforms upward and downward to move the curved seat members in a motion to simulate horseback riding. A first control varies the forward motion of the mobile therapeutic mechanical horse, and a second control varies the seat member cycle speed. The cam and cam shaft are driven by a variable speed, chain-driven motor, enabling adjustable motion and responsiveness to mimic the horse's hip movement and can be adjusted for a walk or a trot movement.

According to other aspects of the present disclosure, the mobile therapeutic mechanical horse may include one or more of the following features. The elevated platform may be capable of movement in sync with the variable speed motion, raising and lowering each curved seat member separately to enhance the sensory therapeutic experience. The mobile therapeutic mechanical horse may be driven by a chain system, equipped with variable control to allow for forward and backward movement. The mobile therapeutic mechanical horse may travel in a circle as would a live horse in a round pen.

One aspect of the invention is directed to a mobile therapeutic mechanical horse to mimic the hip movements of a horse for horseback sensory therapy, the mechanical horse including a steering assembly, a motion assembly, a drive motor, a frame, a mechanical horse body for sitting of a patient and a control system. The steering assemble includes two spindles, a spindle linkage, an actuator arm, an actuator, an actuator rear mounting and two hub mounted rubber tires. The motion assembly includes two pillow blocks, a chain sprocket, a cam and a cam shaft, two bearings, a 12-volt motor with chain sprocket and a roller chain. The drive motor includes a hub chain sprocket attached to rear axil, a 12-volt motor with chain sprocket, a roller chain and two hub mounted rubber tires. The frame includes a rectangular horizontal frame having a length and a center axis along the length and a split rectangular platform having a first and second rotatable platform section, the first rotatable platform having a first hinge attached along the center axis and the second rotatable platform having a second hinge attached along the center axis. The mechanical horse body includes a vertical square tube frame having a horizontal member positioned along the frame length, two curved sheets, each curved sheet having a body hinge attached to the horizontal member, two legs hinged on each of the two curved sheets along curved sheet outer corners, the two legs on each of the two curved sheets pivotally secured to the platform and foam padding disposed on each of the curved sheets with a saddle pad secured to the foam padding and curved sheets. The control system includes a lithium battery and charger, a junction box, 12 and 14 gauge wiring, small plastic housing, two rheostat controls, a three position self-centering toggle switch, an On/Off switch and a fall-safe tethered electrical disconnect. The mobile therapeutic mechanical horse is capable of supporting over 300 pounds and is drivable. The elevated platform is for riding or seating. The control panel includes variable speed control for the mimicked horses hip movement. The two curved sheets may be fiberglass molds of a horse body.

Horseback sensory therapy is an effective treatment modality for treating individuals with mental illness and neurological disorders; however, it is limited to those who are physically capable and willing to ride a horse to engage in traditional horseback sensory therapy. Additionally, there are inherent risks associated with working with live animals, which can further restrict access to this form of therapy. To address these barriers, we present the development of a mobile mechanical therapeutic horse designed to simulate horse movements, thereby providing the benefits of horseback sensory therapy to a broader range of individuals. This innovation aims to make horseback therapeutic benefits accessible to all, particularly those who are unable or unwilling to get on a horse.

Barriers and Issues-Horseback sensory therapy has valuable sensory therapeutic benefits for individuals suffering from mental health and neurological challenges, including depression, anxiety, fear, post-traumatic stress disorder, lack of focus, issues with impulse control, and others. The unique interaction between humans and horses can provide profound support, making this sensory therapy a valuable tool for many seeking relief from these core treatment issues.

Many individuals today are living in a chronic state of stress or in a “fight or flight” response, which can lead to a neurochemical imbalance in the brain and disruption of the vestibular system. This ongoing stress response creates a cascade of physiological effects, including impaired cognitive function, emotional dysregulation, and difficulty learning. The vestibular system, which plays a key role in balance, coordination, and spatial awareness, is particularly vulnerable to such stressors, and when compromised, it can hinder the body's ability to function optimally.

Horse Sensory Therapy utilizes a precise, targeted horse movement to initiate a catalytic effect that enhances the flow of information and optimizes brain function. The movement serves to reset and repair neural pathways damaged by traumatic experiences or chronic stress, effectively neutralizing the body's stress response. By balancing the sympathetic and parasympathetic nervous systems, this sensory therapy facilitates a transition into or boosts Alpha brain wave activity, a state associated with relaxation and cognitive clarity.

As a result, the sensory therapy promotes the repair of the vestibular system and facilitates the opening of the blood-brain barrier, allowing for the free flow of nutrients and information to the brain. With an intact vestibular system, reduced inflammation (as a result of lowered stress), and improved brain function, individuals experience enhanced cognitive abilities, including better retention of new information, improved reasoning and rationalization, increased emotional regulation, and greater overall calmness and relaxation. These improvements lead to heightened productivity, improved decision-making, and a general sense of well-being.

Limitations and Safety Concerns-This form of sensory therapy is inherently limited to those who can physically mount and ride a horse. Many individuals face significant barriers that prevent their participation, including physical disabilities, severe anxiety, or prior traumatic experiences with animals. As a result, a substantial segment of the population remains unable to access the therapeutic benefits that horseback sensory therapy can offer.

Additionally, there are inherent risks associated with working with live animals, such as the potential for falls or other accidents that could lead to injuries. These risks not only pose safety concerns but also deter some individuals from pursuing this form of sensory therapy, even when they might benefit from it. The reliance on live animals further complicates the accessibility of this sensory therapeutic approach, making it necessary to explore alternative solutions.

Furthermore, the effectiveness of horseback sensory therapy is limited by the inherent weight restrictions associated with both the horses and the person conducting the session. Horses have specific weight limits to ensure their safety and well-being, while those providing the sensory therapy also have to adhere to safety protocols that may pose additional restrictions. Consequently, these limitations disqualify a significant number of individuals who could benefit from the therapeutic effects of horseback sensory therapy.

The exclusion of patients having access to horseback sensory therapy poses a critical challenge, as it prevents access to a valuable resource for many individuals who might otherwise find healing through this method. There is an urgent need to address these barriers and expend the reach of horseback sensory therapy so that it can reach a broader audience and provide its benefits to all who need it. It is crucial to develop innovative alternatives that can replicate the beneficial effects of this sensory therapy. A mobile therapeutic mechanical horse that mimics the movements of a horse's hip could serve as a solution, allowing individuals who cannot or are unwilling to ride a horse to still receive the therapeutic advantages of this approach. This innovation holds the potential to bridge the gap and ensure that more individuals can access essential and natural horseback sensory therapy support.

The device is a mobile therapeutic mechanical horse which addresses the limitations associated with traditional horseback sensory therapy. It is specifically designed to mimic the hip movements of a horse, providing an innovative solution for horseback sensory therapy.

The mobile therapeutic mechanical horse is capable of supporting over 300 pounds, features a drivable design, elevated platform for “riding” or seating, and variable speeds for the mimicked horses hip movement. All to enhance the sensory therapeutic experience.

A 12-Volt actuated steering enables precise left and right directional control to maintain the needed circle. The steering assembly enhances maneuverability, making it adaptable to various environments.

A 12-Volt variable speed drive allows the mobile therapeutic mechanical horse to move forward and backward, with the ability to adjust speeds as needed. This allows the adjustment to the precise speed each individual needs during their sensory therapy sessions.

A 12-Volt variable speed motion assembly to replicates the up-and-down hip movement characteristic of a horse. The variable speed adjustment then can meet the specific sensory therapeutic needs. The variable motion is crucial for providing a realistic and beneficial sensory experience. Once the speed is set, it provides consistent and sustained motion.

An elevated platform serves as a substitute for a horse's back for “riding”, allowing individuals to sit as they would if riding a live horse. The mobile therapeutic mechanical horse is capable of movement in sync with the variable speed motion, this platform raises and lowers each half separately enhancing the sensory therapeutic experience. Additionally, it is removable and can be replaced with a chair, accommodating individuals with different needs.

Rubber tires offer a smoother ride, essential for ensuring comfort during sensory therapy sessions. The mobile therapeutic mechanical horse is easily maneuvered in various settings.

Unlike live horses, the mobile therapeutic mechanical horse can accommodate a wide range of body sizes and weights. This adaptability ensures that a more diverse population can benefit from the sensory therapy.

Safety features are provided to protect individuals during sensory therapy sessions including padded surfaces, stability controls, a power-off switch, a fall-safe tethered electrical disconnect, and a locking disc brake to secure the unit while individuals are mounting. The low-profile design keeps individuals less than four feet off the ground, minimizing the risk of falls or injuries.

The mobile therapeutic mechanical horse provides an inclusive and effective solution for horseback sensory therapy, enabling individuals who cannot engage in traditional horseback sensory therapy to experience its numerous benefits.

The system operates on a 12-volt electrical framework, providing a reliable and quiet power source for its various components. The electronic actuator facilitates precise left and right steering. This allows for smooth and controlled navigation in the needed circle.

At the heart of the device's movement is a fabricated cam shaft, which raises and lowers each half of the platform using roller bearings positioned at its ends. This cam shaft is driven by a variable speed, chain-driven motor, enabling adjustable motion and responsiveness. This mimics the horses hip movement and can be adjusted for a walk or a trot movement.

Attached to the platform are square tubing to represent the horse's legs. These legs connect to a frame covered in metal sheeting, representing the body of the horse. As the system operates, the coordinated movement of the legs and body reflects the natural motion of a horse's hips, enhancing the individuals experience.

Additionally, the entire mechanism is driven by a chain system, equipped with variable control to allow for forward and backward movement. This enables the unit to move in a circle as would a live horse in a round pen. All of this gets the individual into their alpha brain wave.

For participant comfort and safety, the horse body is enveloped in one-inch foam and adorned with a saddle blanket. Looking ahead, the final model will feature a fiberglass mold of a horse body, positioned lower to the ground to further improve accessibility and realism. This innovative design not only enhances functionality but also enriches the overall experience for individuals.

There is currently no other device that replicates the specific movement of a horse's hip in a mobile, accessible form that provides the therapeutic benefits of sensory therapy. Currently, the only option for this type of therapy involves live horses. This approach presents challenges for individuals who are fearful of horses, exceed weight limitations (e.g., over 225 pounds), or have physical limitations that prevent mounting or riding.

The mechanical device addresses these challenges by creating a controlled, safe environment that eliminates risks associated with live animals. The unit accommodate individuals weighing over 300 pounds and allows user to sit in a riding position or in a chair. Additionally, it provides consistent, rhythmic movement that enhances the therapeutic experience while offering increased safety, accessibility, and a wider range of benefits for individuals of all abilities.

In a method of using the mobile therapeutic mechanical horse, the individual mounts the mechanical horse in a backward-facing position. A 45 to 55 foot diameter circle is preferred. The device may travel counter-clockwise around the circle at speeds of up to 4 miles per hour. The procedure includes completing several laps, followed by a pause, then repeating this sequence a few times. Specific drills or exercises may be conducted during each pause. This process helps the individual achieve an alpha brain wave state. Reaching this state promotes a calm, peaceful mind and enhances communication pathways in the brain. With the specific cadence and circular path, it works to reduce stress and repair the vestibular system. Typically, ten individual sessions are needed.

According to an aspect of the present disclosure, a mobile therapeutic mechanical horse is provided to mimic the hip movements of a horse for horseback sensory therapy. The mechanical horse comprises a rectangular horizontal frame having a length and a center axis along the length. The mechanical horse includes a split rectangular platform having a first rotatable platform section and second rotatable platform section. The first rotatable platform has a first hinge attached to a frame center member which is positioned along the center axis and the second rotatable platform has a second hinge attached to a frame center member which is attached along the center axis, whereby the first rotatable platform section and second rotatable platform section move consecutively in an arc of about 1 to 10 degrees. A non-slip pad is provided on each of the first rotatable platform section and the second rotatable platform section. The mechanical horse includes a mechanical horse body for sitting of a person. The mechanical horse body includes a front vertical support tube and a rear vertical support tube. A top horizontal member is secured to upper portions of the front vertical support tube and the rear vertical support tube parallel to the frame length. Two curved sheets are provided, each curved sheet having a body hinge attached to the top horizontal member. Two rear legs and two front legs are included, each leg hinged on a top end to one of the two curved sheets along curved sheet outer corners, wherein each of the two rear legs and two front legs have a bottom linkage pivotally secured to the first rotatable platform section or the second rotatable platform section platform. Foam padding is disposed on each of the curved sheets with a saddle pad secured to the foam padding. A motion assembly includes a first cam for vertical movement of a left frame member wherein the left frame member is attached to the bottom surface of the first rotatable platform section and the first cam is attached to a cam shaft, wherein the cam shaft is rotatably secured to the primary body frame by pillow blocks. A second cam on the opposite platform is used similar to the first cam, the second cam being for vertical movement of a right frame member wherein the right frame member is attached to the bottom surface of the second rotatable platform section and the second cam is attached to the cam shaft. A drive chain mechanically links a chain sprocket with a cam sprocket for driving the cam shaft. A steering assembly includes a spindle, a spindle linkage, an actuator arm for moving the spindle linkage about the spindle, an actuator controlled by a left/right direction switch, an actuator rear mount, and two hub mounted rubber tires. A 12-volt drive motor for driving the wheels powers a control panel. A drive system includes a drive motor, a hub chain sprocket attached to rear axle, and two hub mounted rubber tires. A control panel includes a panel housing, a speed rheostat for controlling the speed of the mobile therapeutic mechanical horse, a motion rheostat for controlling the frequency of vertical stroke of the curved sheets, a remote three position self-centering toggle switch for forward, reverse and neutral drive settings, an On/Off switch, the left/right direction switch for setting the front wheels in a straight, right or left direction, and a fall-safe tethered electrical disconnect. A battery is provided which is secured by a battery support which is attached to a portion of the frame, wherein the battery is electrically attached to the control panel. During the vertical stroke of the curved sheets, the curved sheets mimic the hip movement of a horse.

According to other aspects of the present disclosure, the mobile therapeutic mechanical horse may include one or more of the following features. A transmission may be used to maintain a gear ratio selected for proper upper and lower motion speed limits. A battery charger may be provided separate or integrated into the control panel. An optional rotatable carrying handle may be provided. A control panel vertical support and a control panel rotatable support may be included. A junction box may be provided exterior to the control panel to provide easier access to control wires. An optional GPS system may be integrated into the control panel to track the position of the mobile therapeutic mechanical horse, for logging of progress and for safety features. The two curved sheets may be fiberglass molds of a horse body. The battery support may be attached to the top horizontal member.

These and other objects, features, and advantages of the present invention will become more readily apparent from the attached drawings and the detailed description of the preferred embodiments, which follow.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 illustrates a top rear perspective view of a mechanical horse, according to aspects of the present disclosure;

FIG. 2 illustrates a perspective view of a steering assembly for the mechanical horse of FIG. 1;

FIG. 3 illustrates a top rear perspective view of a motion assembly for the mechanical horse of FIG. 1;

FIG. 4 illustrates a perspective view of a control system for the mechanical horse of FIG. 1;

FIG. 5 illustrates a side perspective view of the mechanical horse of FIG. 1;

FIG. 6 illustrates a rear perspective view of the mechanical horse of FIG. 1; and

FIG. 7 illustrates a front perspective view of the mechanical horse of FIG. 1.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The present disclosure relates to a mobile therapeutic mechanical horse designed to mimic the hip movements of a live horse for horseback sensory therapy. This device provides a controlled and adjustable therapeutic experience that simulates horseback riding, offering potential benefits for individuals undergoing sensory therapy. The mobile therapeutic mechanical horse may be used in various settings, including but not limited to therapy centers, rehabilitation facilities, or other suitable locations where sensory therapy is conducted.

In some cases, the mobile therapeutic mechanical horse may include features that allow for precise control of movement, speed, and direction. These features may enable therapists or operators to tailor the therapeutic experience to the specific needs of each patient. The device may be capable of moving in a circular pattern, similar to a live horse in a round pen, while also providing adjustable up-and-down hip movements that replicate the motion experienced during horseback riding.

The mobile therapeutic mechanical horse may incorporate various components and systems to achieve its therapeutic functionality. These components may include, but are not limited to, steering assemblies, drive systems, motion control mechanisms, and seating arrangements. The specific configurations and arrangements of these components may vary across different embodiments of the device, which will be described in further detail in subsequent sections of this disclosure.

In some cases, the mobile therapeutic mechanical horse may offer adjustable settings for speed, motion intensity, and movement patterns. These adjustable features may allow for customization of the sensory therapy session to accommodate different patient needs, skill levels, or therapeutic goals. The device may be designed to provide a safe and controlled environment for sensory therapy, potentially offering benefits similar to those derived from traditional horseback riding therapy.

Multiple embodiments of the mobile therapeutic mechanical horse will be described in the following sections, detailing various aspects of its design, functionality, and potential applications in sensory therapy settings.

In some cases, the mobile therapeutic mechanical horse may include a horizontal frame as part of its basic structure. The horizontal frame may have a length, and along each side of this frame length, a wheel and rubber tire may be positioned. This configuration may provide stability and mobility to the device, allowing it to move in various directions as needed during sensory therapy sessions.

The mobile therapeutic mechanical horse may also feature an elevated platform as part of its design. In some cases, this elevated platform may include a pair of flat metal platforms. These flat metal platforms may be hinged along their adjacent edges, allowing for controlled movement and flexibility in the device's operation. The hinged connection between the flat metal platforms may contribute to the device's ability to simulate the motion of horseback riding.

The combination of the horizontal frame with wheels and the elevated platform with hinged metal platforms may form the foundation of the mobile therapeutic mechanical horse's structure. This basic configuration may allow for the integration of additional components and mechanisms that enable the device to provide a therapeutic experience similar to horseback riding.

In some cases, the mobile therapeutic mechanical horse may incorporate a seating mechanism designed to accommodate patients during sensory therapy sessions. This seating mechanism may be positioned on or above the elevated platform, allowing patients to experience the simulated horseback riding motion in a controlled and safe manner.

The mobile therapeutic mechanical horse may also include basic motion control features. These features may allow for adjustments to the device's movement, potentially including forward and backward motion, directional control, and the simulation of various horse gaits such as walking or trotting. The specific implementation of these motion control features may vary depending on the particular embodiment of the device.

In some cases, the mobile therapeutic mechanical horse may incorporate an enhanced motion control system to provide a more sophisticated and adjustable therapeutic experience. This advanced system may include a 12-volt variable speed drive and a 12-volt variable speed motion control, which work in tandem to offer precise control over the device's movements. This mobile therapeutic mechanical horse may also be propelled by fluids, air, gases, or fuels.

The 12-volt variable speed drive may allow the mobile therapeutic mechanical horse to move forward and backward with adjustable speeds. This feature may enable therapists or operators to fine-tune the device's linear motion to match the specific needs of each patient during their sensory therapy sessions. The ability to control the forward and backward movement may help create a more realistic simulation of horseback riding, potentially enhancing the therapeutic benefits of the treatment.

In some cases, the 12-volt variable speed motion control may be used to regulate the up-and-down hip movement characteristic of horseback riding. This control mechanism may allow for precise adjustments to the intensity and frequency of the simulated horse's gait. By offering variable control over the vertical motion, the device may be able to accommodate patients with different sensory needs or physical capabilities.

The combination of the variable speed drive and motion control may enable the mobile therapeutic mechanical horse to simulate different horse gaits. In some cases, the motion can be adjusted to mimic a walk or a trot movement. This versatility may allow therapists to progressively increase the intensity of the sensory therapy as patients become more comfortable or to tailor the experience to specific therapeutic goals.

The enhanced motion control system may contribute to a more immersive and effective sensory therapy experience. By providing precise control over both the linear and vertical movements of the device, the operator or therapists may be able to create a more customized and responsive therapeutic environment. This level of control may potentially lead to improved outcomes for patients undergoing horseback sensory therapy.

In some cases, the 12-volt system used in both the variable speed drive and motion control may offer advantages in terms of power efficiency and safety. The use of a standardized voltage may also contribute to the overall reliability and maintainability of the mobile therapeutic mechanical horse.

In some cases, the mobile therapeutic mechanical horse may incorporate a 12-volt actuated steering assembly that enables precise left and right directional control. This steering assembly may allow the device to maintain motion in a circular pattern, mimicking the movement of a live horse in a round pen.

The 12-volt actuated steering assembly may be integrated into the device's frame and wheel system. This assembly may include components such as an electric motor, gears, and linkages that work together to control the direction of the wheels. The use of a 12-volt system may provide sufficient power for accurate steering while maintaining energy efficiency.

In some cases, the steering assembly may be designed to automatically adjust the angle of the wheels as the mobile therapeutic mechanical horse moves forward. This continuous adjustment may allow the device to maintain a circular path without requiring constant manual input from an operator. The radius of the circular path may be adjustable, potentially allowing for different sizes of circular patterns to accommodate various therapy settings or patient needs.

The ability to travel in a circle may enhance the therapeutic experience by more closely simulating the movement patterns of a live horse in a round pen. This circular motion may provide additional sensory input for patients undergoing therapy, potentially contributing to improved balance, coordination, and spatial awareness.

In some cases, the circular motion capability may be combined with the variable speed drive and motion control systems. This integration may allow the operator or therapists to adjust not only the speed and intensity of the simulated horseback motion but also the size and direction of the circular path. The combination of these features may provide a highly customizable therapeutic experience that can be tailored to individual patient needs.

The mobile therapeutic mechanical horse's ability to travel in a circle may also offer practical benefits in sensory therapy settings. For example, it may allow the device to be used in smaller spaces compared to a linear path, potentially increasing its versatility in various therapeutic environments.

In some cases, the mobile therapeutic mechanical horse may incorporate an advanced cam and cam shaft assembly driven by a variable speed, chain-driven motor. This sophisticated system may enable more precise control over the simulated horseback riding motion, potentially enhancing the therapeutic experience for patients undergoing sensory therapy.

The cam and cam shaft assembly may be designed to consecutively cycle each of the pair of metal platforms upward and downward. This cycling motion may move the curved seat members in a pattern that closely simulates the motion experienced during horseback riding. The use of a cam and cam shaft mechanism may allow for smooth and controlled transitions between the upward and downward movements, potentially providing a more natural and comfortable experience for the patient.

In some cases, the variable speed, chain-driven motor may offer several advantages in terms of motion control and adjustability. The chain drive system may provide efficient power transmission from the motor to the cam and cam shaft assembly. This efficiency may contribute to the overall responsiveness and precision of the simulated horseback riding motion.

The variable speed feature of the motor may allow for fine-tuning of the motion intensity and frequency. This adjustability may enable the operator or therapists to customize the therapeutic experience based on individual patient needs or therapy goals. For example, the speed may be adjusted to simulate different horse gaits, such as a slow walk or a more vigorous trot.

The mobile therapeutic mechanical horse may be driven by a chain system, which may extend beyond just powering the cam and cam shaft assembly. This chain system may also be responsible for the forward and backward movement of the entire device. The use of a chain drive for both the simulated riding motion and the device's mobility may provide a cohesive and integrated system for overall motion control.

In some cases, the chain-driven system may be equipped with variable control mechanisms. These controls may allow for precise adjustments to both the forward and backward movement of the mobile therapeutic mechanical horse and the intensity of the simulated horseback riding motion. This level of control may enable the operator or therapists to create highly customized sensory therapy sessions tailored to each patient's specific needs and progress.

The combination of the advanced cam and cam shaft assembly with the variable speed, chain-driven motor may result in a highly responsive and adjustable therapeutic device. This system may offer enhanced motion and responsiveness, potentially providing a more immersive and effective sensory therapy experience that closely mimics the movements of a live horse.

In some cases, the mobile therapeutic mechanical horse may be designed as a fully integrated therapeutic system, incorporating all the advanced features previously described to provide a highly customizable and effective sensory therapy experience. This comprehensive embodiment may combine enhanced motion control, circular movement capability, and the advanced cam assembly into a cohesive and sophisticated therapeutic device.

The integrated system may feature the 12-volt variable speed drive for precise control over forward and backward movement, allowing for adjustable speeds to match patient needs during sensory therapy sessions. This may be complemented by the 12-volt variable speed motion control, which may regulate the up-and-down hip movement characteristic of horseback riding. Together, these components may enable the device to simulate various horse gaits, from a gentle walk to a more vigorous trot, providing a range of therapeutic intensities.

The 12-volt actuated steering assembly may be incorporated into this integrated system, enabling the mobile therapeutic mechanical horse to maintain motion in a circular pattern. This feature may allow the device to mimic the movement of a live horse in a round pen, potentially enhancing the sensory input and therapeutic benefits for patients.

At the core of this integrated system, the advanced cam and cam shaft assembly, driven by a variable speed, chain-driven motor, may provide precise control over the simulated horseback riding motion. This mechanism may consecutively cycle each of the pair of metal platforms upward and downward, moving the curved seat members in a pattern that closely replicates the motion experienced during horseback riding.

In some cases, the elevated platform may be designed to raise and lower each curved seat member separately. This independent movement of the seat members may enhance the sensory therapeutic experience by providing a more nuanced and realistic simulation of horseback riding. The separate movement of each seat member may allow for a more dynamic and responsive therapy session, potentially accommodating patients with different physical needs or therapy goals.

The chain system that drives the mobile therapeutic mechanical horse may be equipped with variable control mechanisms, allowing for precise adjustments to both the device's mobility and the intensity of the simulated riding motion. This level of control may enable therapists to create highly personalized sensory therapy sessions tailored to each patient's specific requirements and progress.

By integrating these advanced features into a single, comprehensive system, the mobile therapeutic mechanical horse may offer a highly adaptable and responsive therapeutic tool. Therapists may have the ability to adjust multiple parameters simultaneously, potentially creating a more immersive and effective sensory therapy experience. For example, the circular movement, speed, and intensity of the simulated riding motion may all be fine-tuned in real-time to match the patient's comfort level and therapeutic objectives.

The fully integrated system may also offer improved synchronization between different components. For instance, the variable speed drive controlling forward and backward movement may be coordinated with the cam assembly's cycling of the seat members, potentially resulting in a more natural and fluid simulation of horseback riding.

In some cases, this comprehensive embodiment may include additional features to enhance the overall therapeutic experience. These may include safety mechanisms, ergonomic adjustments for different patient sizes, or interfaces for the operator or therapists to easily control and monitor the various aspects of the device's operation.

The mobile therapeutic mechanical horse, when configured as a fully integrated therapeutic system, may provide a versatile and powerful tool for sensory therapy. By combining advanced motion control, circular movement capability, and sophisticated seat member actuation, the device may offer a highly customizable experience that closely mimics the therapeutic benefits of horseback riding in a controlled and safe environment.

In some cases, the mobile therapeutic mechanical horse may incorporate various additional features and variations to enhance its therapeutic applications and user experience. These modifications may be designed to accommodate a wider range of patients, improve safety, or provide more precise control over the therapy session.

One potential variation may include an adjustable seating system. This system may allow for modifications to the seat height, width, or angle to accommodate patients of different sizes or with specific physical needs. In some cases, the adjustable seating may include interchangeable seat cushions with varying levels of firmness or contour to provide optimal comfort and support for different patients.

Safety features may be incorporated into the mobile therapeutic mechanical horse to ensure patient well-being during sensory therapy sessions. In some cases, these safety features may include harnesses or restraints that secure the patient to the seat while allowing for the necessary range of motion during the simulated horseback riding experience. The harnesses may be adjustable to fit patients of various sizes and may be designed with quick-release mechanisms for easy removal if needed.

In some cases, the mobile therapeutic mechanical horse may be equipped with a digital control interface. This interface may allow therapists or operators to precisely adjust and monitor various aspects of the device's operation. The digital control system may include touchscreen displays or mobile device integration, enabling real-time adjustments to speed, motion intensity, and movement patterns. In some cases, the digital interface may also provide data logging capabilities, allowing therapists to track patient progress over time and adjust treatment plans accordingly.

Additional sensory elements may be incorporated into the mobile therapeutic mechanical horse to further enhance the therapeutic experience. In some cases, these elements may include audio systems that produce horse-like sounds or nature ambiance to create a more immersive environment. Tactile feedback mechanisms may be integrated into the seat or other contact points to simulate the feeling of a horse's coat or the sensation of riding outdoors.

In some cases, the mobile therapeutic mechanical horse may feature modular components that allow for easy customization or upgrades. This modular design may enable therapy centers to adapt the device to evolving patient needs or incorporate new therapeutic technologies as they become available.

The mobile therapeutic mechanical horse may also be designed with energy efficiency in mind. In some cases, the device may incorporate regenerative braking systems or solar charging capabilities to extend operating times and reduce energy consumption.

Accessibility features may be added to the mobile therapeutic mechanical horse to accommodate patients with various physical abilities. In some cases, these features may include assisted mounting systems, such as lifts or ramps, to help patients safely board the device. Adjustable handholds or support bars may be incorporated to provide additional stability for patients who require it.

In some cases, the mobile therapeutic mechanical horse may be equipped with telemetry systems that allow for remote monitoring and control. This feature may enable therapists to supervise sessions from a distance or provide guidance to caregivers administering therapy at home.

The mobile therapeutic mechanical horse may also incorporate programmable therapy routines. In some cases, these pre-set programs may simulate specific riding experiences or target particular therapeutic goals, allowing for consistent and repeatable therapy sessions.

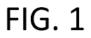

FIG. 1 shows a mobile therapeutic mechanical horse 100 to mimic the hip movements of a horse for horseback sensory therapy. The mechanical horse includes a steering assembly and a motion assembly. The mobile therapeutic mechanical horse includes a drive motor assembly having a drive motor 116 and a chain 118 attaching a sprocket on the drive motor 116 and on the shaft for driving the tire rotation. The mobile therapeutic mechanical horse 100 includes a frame 150, a mechanical horse body 102 for sitting of a patient and a control system 190.

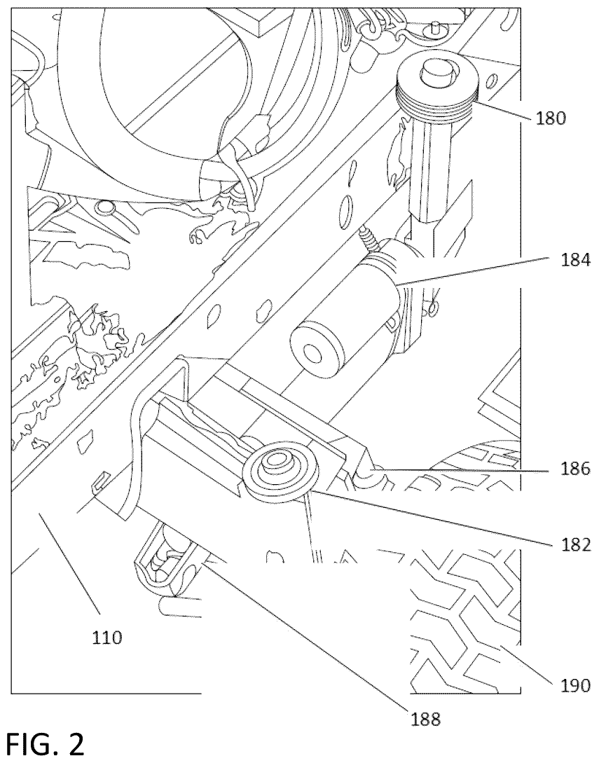

The steering assembly shown in FIG. 2 is mounted on the front of the mobile therapeutic mechanical horse and includes two spindles 182, a spindle linkage 186, an actuator arm 188, an actuator 184, an actuator rear mounting 180 and two hub mounted rubber tires 190. Two additional hub mounted rubber tires are rotatably mounted on the rear of the mobile therapeutic mechanical horse.

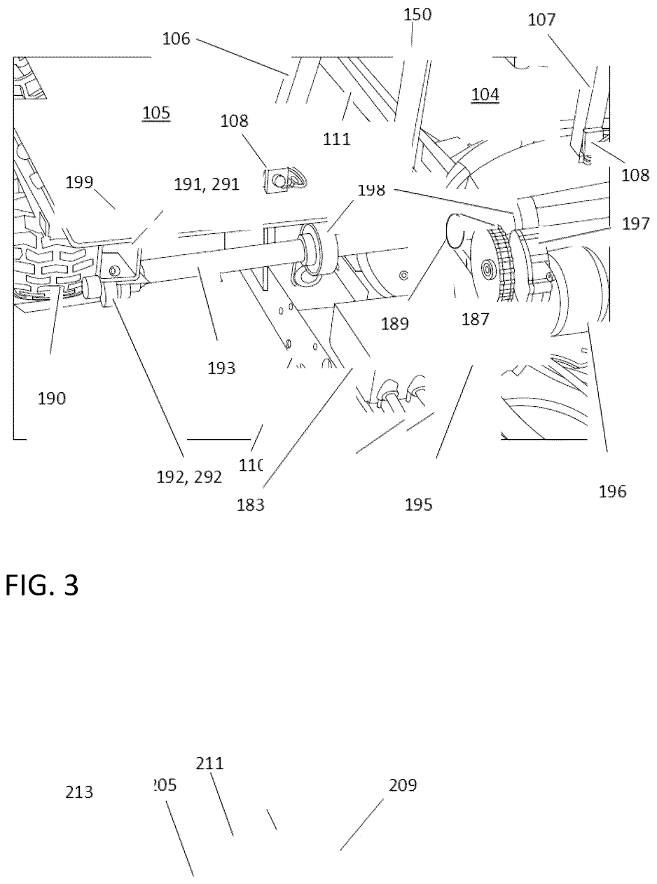

The motion assembly shown in FIG. 3 includes two pillow blocks 198, a chain sprocket 189, a cam shaft 193, two bearings 192, 292, a 12-volt motor with chain sprocket 196 and a roller chain 195.

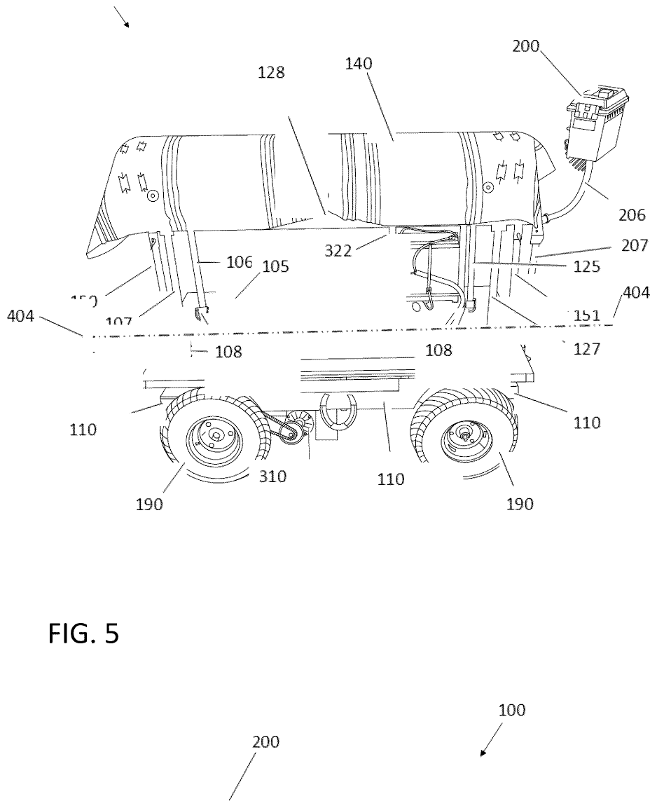

The drive motor assembly shown in FIG. 5 includes a hub chain sprocket (not shown) attached to rear axil (not shown), a 12-volt motor with chain sprocket 300, a roller chain 310 and two hub mounted rubber tires 190. The frame 110 includes a rectangular horizontal frame having a length and a center axis along the length.

A split rectangular platform shown in FIG. 5 includes a first and second rotatable platform section 104. The first rotatable platform has a first hinge attached along a frame center member 111 which is positioned along the center axis and the second rotatable platform having a second hinge attached along the center axis. The mechanical horse body includes a vertical square tube frame having a horizontal member positioned along the frame length, two curved sheets, each curved sheet having a body hinge attached to the horizontal member, two legs 106 (FIG. 1) hinged with hinges 170 on each of the two curved sheets 102 along curved sheet outer corners, the two legs 106 on each of the two curved sheets pivotally secured by a joint 108 to the platform and foam padding 130 disposed on each of the curved sheets with a saddle pad 140 secured to the foam padding and curved sheets.

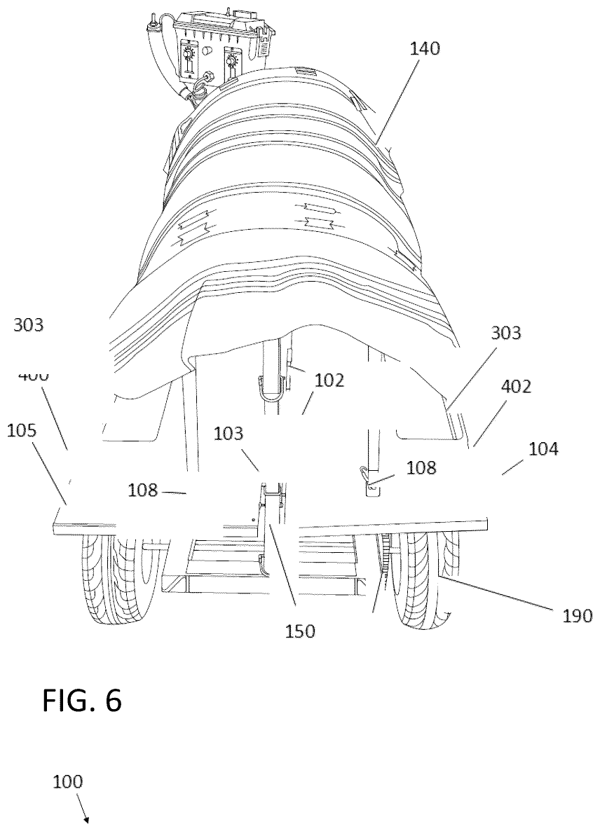

The control system shown in FIG. 6 includes a lithium battery and charger 320, a junction box 183, 12, 14 or other gauge wiring, a housing 202, a rheostat control 212 for controlling speed of the motor 116 and a rheostat control 204 for controlling speed of the motion controlling the horse hip simulation. The control system includes a three position self-centering toggle switch 210 for controlling left and right motion of the mobile therapeutic mechanical horse. An On/Off switch 208 controls power to the control system and a fall-safe tethered electrical disconnect 213 cuts power to the control system or motor if the operator was to slip or fall and pull the tethered stitch. The mobile therapeutic mechanical horse is capable of supporting over 300 pounds and is drivable. The elevated platform is for riding or seating, and the control panel includes variable speed control for the mimicked horses hip movement. The two curved sheets may be fiberglass molds of a horse body. A patient handle may be provided which may cave the shape of a saddle horn. A step may be placed on the platform for a patient to use to mount the mobile therapeutic mechanical horse.

Referring to FIGS. 1-7, a mechanical horse 100 provides a therapeutic device that simulates equine movement for horseback sensory therapy applications. The mechanical horse 100 eliminates risks associated with live animals while delivering controlled, rhythmic movement patterns that replicate the hip movements of a horse. This therapeutic approach addresses limitations of traditional horseback therapy by providing increased safety, accessibility, and consistent movement patterns for individuals with mental illness and neurological disorders.

The mechanical horse 100 accommodates individuals weighing over 300 pounds and allows users to sit in a riding position or in a chair configuration. As shown in FIG. 1, the mechanical horse 100 includes a rectangular horizontal frame 110 that serves as the primary structural foundation. A first rotatable platform section 104 and a second rotatable platform section 105 form a split platform arrangement, with the first rotatable platform section 104 connected to the rectangular horizontal frame 110 through a first hinge 102 and the second rotatable platform section 105 connected through a second hinge 103. The first rotatable platform section 104 and second rotatable platform section 105 move in an arc of about 1 to 10 degrees during operation.

With continued reference to FIG. 1, the mechanical horse 100 incorporates a motion assembly 116 that generates the therapeutic movement patterns. Two curved sheets 126, 128 form the riding surface and connect to a top horizontal member 122 through body hinges 170. The curved sheets 126, 128 include fiberglass molds of a horse body that provide anatomically accurate contours for the therapeutic experience. Foam padding 130 covers each of the curved sheets 126, 128, and a saddle pad 140 secures to the foam padding 130 to enhance user comfort during therapy sessions.

The structural framework includes a front vertical support tube 150 and rear legs 106, 107 that provide vertical support for the curved sheets 126, 128. As shown in FIG. 5, front legs 125, 127 and a rear support tube 151 complete the support structure. Bottom linkages 108 connect the rear legs 106, 107 and front legs 125, 127 to the first rotatable platform section 104 and second rotatable platform section 105, enabling coordinated movement between the platform sections and the riding surface. The mechanical horse 100 includes rubber tires 190 mounted to a rear axle 310 for mobility during therapy sessions.

As further shown in FIG. 3, the motion assembly 196 incorporates a cam-driven system that creates the vertical movement patterns. A first cam 192 connects to a left frame member 191 that attaches to the bottom surface of the first rotatable platform section 104, while a second cam 292 connects to a right frame member 291 that attaches to the second rotatable platform section 105. A cam shaft 193 supports both the first cam 192 and second cam 292, with pillow blocks 198 providing rotational support for the cam shaft 193 within the rectangular horizontal frame 110. A drive chain 187 mechanically links a chain sprocket 195 with the cam shaft 193, and a transmission 197 maintains gear ratios for proper movement speed limits. A motor mount not shown, support bracket not shown, and mounting plate not shown provide structural support for the drive components.

Referring to FIG. 2, a steering assembly enables directional control of the mechanical horse 100 during therapy sessions. The steering assembly includes a spindle 182 connected to a spindle linkage 186, with an actuator arm 188 positioned to move the spindle linkage 186 about the spindle 182. An actuator 184 provides the motive force for steering operations, and an actuator mount 180 secures the actuator 184 to the frame structure. The mechanical horse 100 travels counter-clockwise around a 45-to 55-foot diameter circle at speeds of up to 4 miles per hour during therapeutic applications.

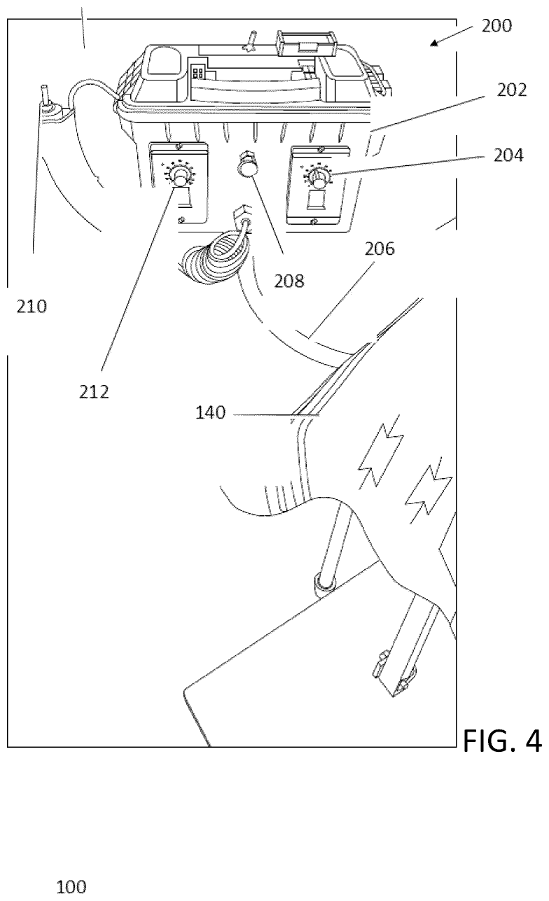

As shown in FIG. 4, a control panel 200 housed within a panel housing 202 provides operational control for the therapeutic device. The control panel 200 includes a motion rheostat 204 for controlling the frequency of vertical movement of the curved sheets 126, 128, and a speed rheostat 212 for controlling the travel speed of the mechanical horse 100. A three-position toggle switch 210 enables left and right turn, and neutral drive settings controls the steering direction. A power switch 208 provides on/off functionality, and an electrical disconnect 213 serves as a safety feature. A GPS system 209 integrates into the control panel 200 to track position, log progress, and provide safety features during therapy sessions.

With reference to FIG. 5, a drive motor 300 powers the mechanical horse 100 and connects to the rear axle 310 through the drive system. A 12-volt battery 320 powers the control panel 200 and drive motor 300, with a battery support 322 securing the battery 320 to the top horizontal member 122 of the rectangular horizontal frame 110. The control panel 200 mounts to the mechanical horse 100 through a vertical support 207 and a rotatable support 206 that enable positioning adjustments. A carrying handle 205 shown in FIG. 4 facilitates transport of the control panel 200, and a junction box 83 shown in FIG. 11 provides electrical connections between system components.

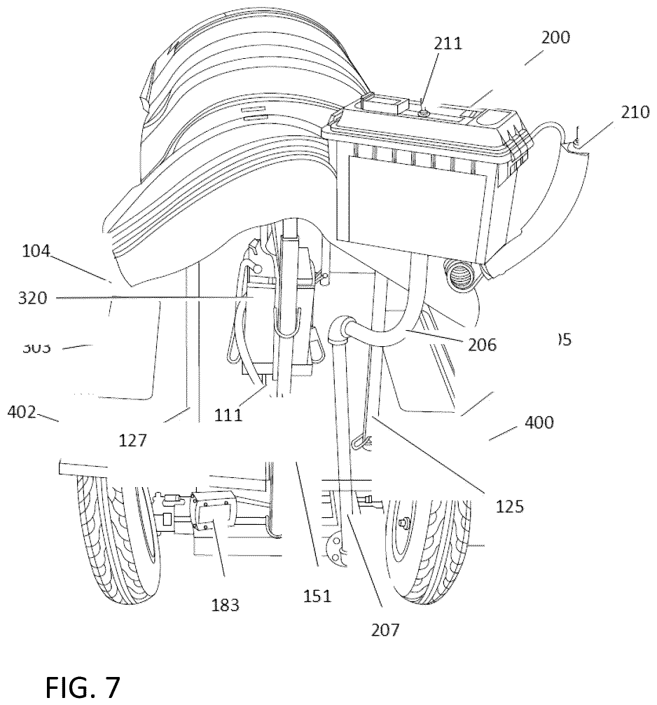

The therapeutic protocol involves mounting the mechanical horse 100 in a backward-facing position, with the therapy procedure including completing several laps around the circular path, followed by a pause, then repeating this sequence with specific drills or exercises conducted during each pause. As shown in FIGS. 6 and 7, non-slip pads 303 on the first rotatable platform section 104 and second rotatable platform section 105 provide secure footing during mounting and dismounting procedures. The therapeutic treatment typically requires ten individual sessions to achieve the desired neurological benefits, including reaching an alpha brain wave state that promotes calm mental states and enhanced neural communication pathways.

Referring to FIG. 1, the rectangular horizontal frame 110 forms the foundational structure of the mechanical horse 100, providing the primary load-bearing platform that supports all operational components. A frame center member 111 along the central axis 404 shown in FIG. 5 extends along the length of the rectangular horizontal frame 110, defining the longitudinal centerline about which the split platform sections operate. The rectangular horizontal frame 110 incorporates robust construction materials and dimensional specifications that enable the mechanical horse 100 to accommodate individuals weighing over 300 pounds while maintaining structural integrity during therapeutic operations. The frame geometry establishes the spatial relationships between the various mechanical assemblies and provides mounting points for the drive systems, motion components, and control elements.

The front vertical support tube 150 extends upward from the rectangular horizontal frame 110 to establish the forward structural anchor point for the riding assembly. The front vertical support tube 150 connects to the top horizontal member 122, which spans the length of the frame structure parallel to the central axis. The top horizontal member 122 serves as the primary mounting interface for the curved riding surfaces and provides the upper structural connection between the front and rear support elements. A rear support tube 151 completes the vertical framework by extending upward from the rear portion of the rectangular horizontal frame 110, creating a rigid structural triangle with the front vertical support tube 150 and the top horizontal member 122.

With continued reference to FIG. 1, two curved sheets 126, 128 attach to the top horizontal member 122 through a body hinge 170 that enables the therapeutic rocking motion characteristic of equine movement simulation. The curved sheets 126, 128 comprise fiberglass molds of a horse body that replicate the anatomical contours and surface geometry of an actual horse. Each of the curved sheets 126, 128 connects to the split platform assembly through a series of mechanical linkages that translate the platform motion into the riding surface movement. A rear leg 106 and a rear leg 107 extend downward from the curved sheets 126, 128 to connect with the first rotatable platform section 104 and the second rotatable platform section 105 through a bottom linkage 108.

As shown in FIG. 5, a front leg 125 and a front leg 127 provide additional connection points between the curved sheets 126, 128 and the rotatable platform sections. The front leg 125 and the front leg 127 work in coordination with the rear leg 106 and the rear leg 107 to create a four-point suspension system that transfers the platform motion to the riding surface. The bottom linkage 108 incorporates pivotal connections that allow the legs to articulate as the first rotatable platform section 104 and the second rotatable platform section 105 move through their operational arc of about 1 to 10 degrees. This articulation creates the characteristic hip movement patterns that simulate the gait of a live horse during therapeutic sessions.

The structural assembly enables users to sit in a riding position or in a chair configuration on the mechanical horse 100, with the frame dimensions and support geometry accommodating various seating arrangements. As further shown in FIG. 3, a pillow block 198 provides rotational support for the cam shaft 193 within the rectangular horizontal frame 110, demonstrating the integration of the motion assembly components with the primary frame structure. A vertical movement indicator 199 illustrates the range of motion generated by the cam-driven system, with the frame structure designed to withstand the cyclical loading patterns created during therapeutic operations. The rectangular horizontal frame 110 also incorporates mounting provisions for rubber tires 190 that enable the mechanical horse 100 to travel counter-clockwise around a 45-to 55-foot diameter circle at speeds of up to 4 miles per hour during therapy sessions.

Referring to FIG. 6 and FIG. 7, a non-slip pad 303 attaches to each of the first rotatable platform section 104 and the second rotatable platform section 105 to provide secure footing during mounting and dismounting procedures. The non-slip pad 303 enhances safety when individuals mount the mechanical horse 100 in a backward-facing position during therapy, which represents the standard positioning protocol for the therapeutic treatment. The frame assembly supports the complete therapy procedure that includes completing several laps around the circular path, followed by a pause, then repeating this sequence with specific drills or exercises conducted during each pause, with the structural components designed to withstand the repetitive loading cycles associated with the typical ten individual sessions needed for the therapy treatment.

Referring to FIG. 1, the split rectangular platform forms the foundational movement system of the mechanical horse 100, comprising the first rotatable platform section 104 and the second rotatable platform section 105 that operate in coordinated motion to generate the therapeutic hip movement patterns. The first rotatable platform section 104 connects to the rectangular horizontal frame 110 through the first hinge 102 on the frame center member 111 which is positioned along central axis 404 shown in FIG. 5, to establish the rotational pivot point for the left side of the platform assembly. The second rotatable platform section 105 connects to the rectangular horizontal frame 110 through the second hinge 103 on the frame center member 111 which is positioned along or adjacent the central axis 404 to create the corresponding rotational pivot point for the right side of the platform assembly. The first hinge 102 and the second hinge 103 enable independent rotational movement of each platform section while maintaining structural connection to the rectangular horizontal frame 110.

The first rotatable platform section 104 and the second rotatable platform section 105 move in an arc of about 1 to 10 degrees during therapeutic operations, creating the controlled angular displacement that translates into the riding surface motion. The rotational movement of the first rotatable platform section 104 operates independently from the second rotatable platform section 105, allowing for alternating motion patterns that simulate the natural gait characteristics of equine movement. The angular range of motion generates sufficient displacement to create therapeutic benefits while maintaining safe operational parameters for individuals weighing over 300 pounds who sit in a riding position or in a chair configuration on the mechanical horse 100.

With continued reference to FIG. 1, the bottom linkage 108 connects the rear leg 106 and the rear leg 107 to the first rotatable platform section 104 and the second rotatable platform section 105, transferring the rotational motion from the platform sections to the riding assembly. The bottom linkage 108 incorporates pivotal connections that accommodate the angular movement of the platform sections while maintaining structural integrity throughout the operational cycle. As shown in FIG. 5, the front leg 125 and the front leg 127 provide additional connection points between the riding assembly and the rotatable platform sections, creating a four-point suspension system that distributes the rotational forces across multiple attachment points.

The rotational movement system enables the mechanical horse 100 to travel counter-clockwise around a 45 to 55 foot diameter circle at speeds of up to 4 miles per hour while simultaneously generating the therapeutic motion patterns. The first rotatable platform section 104 and the second rotatable platform section 105 operate in coordination with the motion assembly 116 to create the combined translational and rotational movement that characterizes the therapeutic protocol. Individuals mount the mechanical horse 100 in a backward-facing position during therapy, with the platform sections providing stable support throughout the therapy procedure that includes completing several laps, followed by a pause, then repeating this sequence with specific drills or exercises conducted during each pause.

As further shown in FIG. 3, the left frame member 191 attaches to the bottom surface of the first rotatable platform section 104, while the right frame member 291 attaches to the bottom surface of the second rotatable platform section 105, establishing the mechanical connection between the cam-driven motion assembly and the rotatable platform sections. The first cam 192 drives the vertical movement of the left frame member 191, which translates into rotational motion of the first rotatable platform section 104 about the first hinge 102. The second cam 292 drives the vertical movement of the right frame member 291, which creates corresponding rotational motion of the second rotatable platform section 105 about the second hinge 103. The cam shaft 193 supports both the first cam 192 and the second cam 292, with the pillow block 198 providing rotational support within the rectangular horizontal frame 110.

Referring to FIG. 6 and FIG. 7, the non-slip pad 303 attaches to each of the first rotatable platform section 104 and the second rotatable platform section 105 to provide secure footing during mounting and dismounting procedures. The non-slip pad 303 maintains surface traction as the platform sections move through their rotational arc, enhancing safety during the therapeutic sessions that typically require ten individual sessions for complete treatment. The rotational movement of the platform sections coordinates with the overall mobility system, including a rubber tire 190 that enables the mechanical horse 100 to traverse the circular therapy path while maintaining the therapeutic motion patterns generated by the split platform system.

Referring to FIG. 1, the curved sheets 126, 128 form the anatomical riding surface of the mechanical horse 100 and replicate the body contours of an actual horse to provide authentic therapeutic positioning for users. A curved sheet 126 and a curved sheet 128 attach to the top horizontal member 122 through the body hinge 170, which enables the therapeutic rocking motion that characterizes equine movement simulation. The curved sheets 126, 128 comprise fiberglass molds of a horse body that reproduce the three-dimensional geometry and surface textures found on live horses, creating realistic tactile feedback during therapeutic sessions. The body hinge 170 establishes the primary pivot point for the riding surface assembly and allows the curved sheets 126, 128 to articulate in response to the motion generated by the underlying platform system.

The foam padding 130 covers each of the curved sheets 126, 128 to provide cushioning and comfort for individuals during extended therapeutic sessions. The foam padding 130 conforms to the contoured surface of the curved sheets 126, 128 while maintaining sufficient thickness to distribute pressure loads across the riding surface. The saddle pad 140 secures to the foam padding 130 and establishes the final riding interface that accommodates users who sit in a riding position or in a chair configuration on the mechanical horse 100. The saddle pad 140 incorporates materials and construction techniques that withstand the loading conditions created by individuals weighing over 300 pounds while maintaining comfort throughout the therapeutic protocol.

With continued reference to FIG. 1, the rear leg 106 and the rear leg 107 extend downward from the curved sheets 126, 128 to establish the primary structural connection between the riding surface and the underlying motion system. The rear leg 106 connects to the first rotatable platform section 104 through the bottom linkage 108, while the rear leg 107 connects to the second rotatable platform section 105 through a corresponding bottom linkage 108. The bottom linkage 108 incorporates pivotal connections that accommodate the angular movement of the first rotatable platform section 104 and the second rotatable platform section 105 as these components move in an arc of about 1 to 10 degrees during therapeutic operations. The pivotal design of the bottom linkage 108 transfers the rotational motion from the platform sections to the curved sheets 126, 128 while allowing for the articulation needed to create realistic hip movement patterns.

As shown in FIG. 5, the front leg 125 and the front leg 127 provide additional structural connections between the curved sheets 126, 128 and the rotatable platform sections, creating a four-point suspension system that distributes mechanical loads across multiple attachment points. The front leg 125 connects to the first rotatable platform section 104, while the front leg 127 connects to the second rotatable platform section 105, with both connections incorporating the same pivotal design found in the bottom linkage 108. The front leg 125 and the front leg 127 work in coordination with the rear leg 106 and the rear leg 107 to translate the alternating motion of the platform sections into the characteristic rocking movement experienced by users on the riding surface. The four-point suspension system maintains structural stability while enabling the curved sheets 126, 128 to follow the motion patterns generated by the underlying cam-driven mechanism.

The leg assembly configuration enables individuals to mount the mechanical horse 100 in a backward-facing position during therapy, with the curved sheets 126, 128 providing stable seating throughout the therapy procedure that includes completing several laps around a circular path, followed by a pause, then repeating this sequence with specific drills or exercises conducted during each pause. As further shown in FIG. 3, the structural connections between the legs and the platform sections coordinate with the motion assembly 116 to create the therapeutic movement patterns that simulate equine gait characteristics. The curved sheets 126, 128 accommodate the loading conditions associated with the mechanical horse 100 traveling counter-clockwise around a 45 to 55 foot or similar diameter circle at speeds of up to about 4 miles per hour while maintaining the comfort and safety features needed for the typical ten individual sessions that comprise the complete therapeutic treatment protocol.

Referring to FIG. 3, the motion assembly 116 incorporates a cam-driven mechanism that generates the therapeutic vertical movement patterns characteristic of equine gait simulation. The cam shaft 193 extends horizontally across the rectangular horizontal frame 110 and serves as the primary rotational element that drives the alternating motion of the platform sections. The first cam 192 mounts to the cam shaft 193 and connects to the left frame member 191, which attaches to the bottom surface of the first rotatable platform section 104. The second cam 292 mounts to the cam shaft 193 at a position offset from the first cam 192 and connects to the right frame member 291, which attaches to the bottom surface of the second rotatable platform section 105. The offset positioning of the first cam 192 and the second cam 292 creates the alternating vertical displacement patterns that translate into the therapeutic rocking motion experienced by users who sit in a riding position or in a chair on the mechanical horse 100.

The pillow block 198 provides rotational support for the cam shaft 193 within the rectangular horizontal frame 110, enabling smooth rotation while maintaining precise alignment of the cam elements. The pillow block 198 incorporates bearing assemblies that accommodate the rotational loads generated during therapeutic operations while supporting individuals weighing over 300 pounds. The drive chain 187 mechanically connects the chain sprocket 195 to the cam shaft 193, transferring rotational power from the drive system to the motion assembly 116. The chain sprocket 195 mounts to the cam shaft 193 and receives rotational input through the drive chain 187, which connects to the drive motor 300 through the transmission 197. The transmission 197 establishes the gear ratios that control the rotational speed of the cam shaft 193, enabling the mechanical horse 100 to generate therapeutic movement patterns while traveling counter-clockwise around a 45-to 55-foot diameter circle at speeds of up to 4 miles per hour.

With continued reference to FIG. 3, the left frame member 191 extends vertically downward from the first cam 192 and transfers the vertical displacement generated by the cam rotation to the first rotatable platform section 104. The right frame member 291 extends vertically downward from the second cam 292 and transfers corresponding vertical displacement to the second rotatable platform section 105. The vertical movement indicator 199 illustrates the range of motion generated by the cam system, with the first rotatable platform section 104 and the second rotatable platform section 105 moving in an arc of about 1 to 10 degrees in response to the cam-driven vertical displacement. The alternating motion of the left frame member 191 and the right frame member 291 creates the characteristic hip movement patterns that simulate the natural gait of a horse, providing therapeutic benefits for individuals who mount the mechanical horse 100 in a backward-facing position during therapy sessions.

The motor mount 196 secures the drive motor 300 to the rectangular horizontal frame 110 and positions the drive motor 300 to interface with the transmission 197 and drive chain 187. The support bracket 194 provides additional structural reinforcement for the motion assembly 116 components and distributes the mechanical loads generated during operation across the rectangular horizontal frame 110. The mounting plate 189 establishes connection points for various motion assembly 116 elements and integrates with the rectangular horizontal frame 110 to create a unified structural system. The coordinated operation of these components enables the motion assembly 116 to generate consistent therapeutic movement patterns throughout the therapy procedure that includes completing several laps, followed by a pause, then repeating this sequence with specific drills or exercises conducted during each pause, with the complete treatment protocol typically requiring ten individual sessions.

As further shown in FIG. 1, the motion assembly 116 integrates with the curved sheets 126, 128 through the mechanical linkages formed by the rear legs 106, 107 and the front legs 125, 127. The vertical displacement generated by the first cam 192 and the second cam 292 transfers through the left frame member 191 and the right frame member 291 to create rotational motion of the first rotatable platform section 104 and the second rotatable platform section 105 about their respective hinges. The bottom linkage 108 accommodates this rotational motion and transfers the movement to the curved sheets 126, 128, which comprise fiberglass molds of a horse body that provide anatomically accurate riding surfaces. The 12-volt battery 320 powers the drive motor 300 and the control panel 200, with the battery support 322 attached to the top horizontal member 122 to provide stable mounting for the electrical power source. The motion assembly 116 operates under control of the motion rheostat 204, which regulates the frequency of the vertical movement patterns generated by the cam system to optimize the therapeutic benefits delivered during each treatment session.

Referring to FIG. 2, the steering assembly provides precise directional control for the mechanical horse 100 during therapeutic operations and incorporates multiple interconnected components that work together to enable controlled navigation around the circular therapy path. The spindle 182 forms the central pivot point of the steering mechanism and establishes the rotational axis about which the directional changes occur. The spindle 182 connects directly to the spindle linkage 186, which transfers the rotational motion from the steering control system to the wheel assembly. The spindle linkage 186 incorporates mechanical connections that translate the angular displacement generated by the steering actuator into corresponding wheel orientation changes that guide the mechanical horse 100 along the prescribed counter-clockwise path around a 45 to 55 foot diameter circle, although the mechanical horse 100 may be used for any diameter circle.

The actuator arm 188 provides the mechanical interface between the steering control system and the spindle linkage 186, positioning to move the spindle linkage 186 about the spindle 182 in response to directional commands from the control panel 200. The actuator arm 188 incorporates leverage ratios that amplify the control inputs and translate them into the angular displacements needed to achieve the steering angles for the circular therapy path. The actuator 184 generates the motive force that drives the steering operations and connects to the actuator arm 188 to provide controlled movement of the steering assembly components. The actuator 184 receives electrical control signals from the direction switch 210 located on the control panel 200, enabling operators to select straight, right, or left directional settings during therapeutic sessions.

With continued reference to FIG. 2, the actuator mount 180 secures the actuator 184 to the structural framework of the mechanical horse 100 and provides stable positioning that maintains alignment between the actuator 184 and the actuator arm 188 throughout the steering operations. The actuator mount 180 incorporates mounting provisions that distribute the mechanical loads generated during directional changes across the rectangular horizontal frame 110, ensuring structural integrity when the mechanical horse 100 accommodates individuals weighing over 300 pounds who sit in a riding position or in a chair configuration. The rubber tire 190 connects to the steering assembly through the spindle 182 and responds to the angular positioning commands generated by the coordinated operation of the actuator 184, actuator arm 188, and spindle linkage 186. The rubber tire 190 provides ground contact and traction during the directional changes that enable the mechanical horse 100 to travel at speeds of up to 4 miles per hour while maintaining the prescribed circular path.

The steering assembly operates in coordination with the drive motor 300 and the motion assembly 116 to create the combined translational and therapeutic movement patterns that characterize the treatment protocol. The directional control system enables individuals who mount the mechanical horse 100 in a backward-facing position during therapy to experience consistent circular motion throughout the therapy procedure that includes completing several laps, followed by a pause, then repeating this sequence with specific drills or exercises conducted during each pause. The actuator 184 receives power from the 12-volt battery 320 through electrical connections that route through the junction box 183, with the GPS system 209 integrated into the control panel 200 providing position tracking and safety monitoring during the steering operations. The steering assembly components coordinate with the first rotatable platform section 104 and the second rotatable platform section 105 as these platform sections move in an arc of about 1 to 10 degrees, ensuring that the directional control functions operate independently from the therapeutic motion patterns generated by the curved sheets 126, 128 that comprise fiberglass molds of a horse body.

As further shown in FIG. 1, the steering assembly integrates with the overall mechanical horse 100 structure through connections to the rectangular horizontal frame 110 and coordinates with the rear axle 310 to provide four-wheel directional control during therapeutic operations. The spindle 182 and the spindle linkage 186 work together to maintain precise steering angles throughout the complete treatment protocol that typically requires ten individual sessions, with the actuator mount 180 providing stable support for the actuator 184 during the repetitive steering cycles associated with the circular therapy path. The steering assembly components accommodate the loading conditions created when the mechanical horse 100 travels counter-clockwise around the therapy circle while simultaneously generating the therapeutic hip movement patterns through the coordinated operation of the motion assembly 116, the curved sheets 126, 128, and the split platform system formed by the first rotatable platform section 104 and the second rotatable platform section 105.

Referring to FIG. 5, the drive motor 300 provides the primary motive force for the mechanical horse 100 and connects directly to the rear axle 310 to deliver rotational power to the mobility system. The drive motor 300 incorporates electrical specifications that enable operation from the 12-volt battery 320, with the motor design optimized to provide sufficient torque for propelling the mechanical horse 100 while accommodating individuals weighing over 300 pounds who sit in a riding position or in a chair configuration. The drive motor 300 mounts to the rectangular horizontal frame 110 through the motor mount 196, which positions the drive motor 300 to interface directly with the transmission 197 and the drive chain 187. The electrical connections between the drive motor 300 and the 12-volt battery 320 route through the junction box 183, with the control panel 200 providing operational control through the speed rheostat 212 and the toggle switch 210 that enables forward, reverse, and neutral drive settings.

The rear axle 310 extends horizontally across the width of the rectangular horizontal frame 110 and serves as the primary load-bearing shaft that supports the weight of the mechanical horse 100 and its occupants during therapeutic operations. As shown in FIG. 6, the rear axle 310 connects to the rubber tires 190 and transfers the rotational power from the drive motor 300 to the ground contact surfaces. The rear axle 310 incorporates bearing assemblies that accommodate the rotational loads generated during operation while supporting the structural loads created when individuals weighing over 300 pounds mount the mechanical horse 100 in a backward-facing position during therapy. The rear axle 310 coordinates with the drive chain 187 through the chain sprocket 195, which mounts to the rear axle 310 and receives rotational input from the transmission 197. The transmission 197 establishes gear ratios that enable the mechanical horse 100 to travel counter-clockwise around a 45-to 55-foot diameter circle at speeds of up to 4 miles per hour while maintaining the therapeutic motion patterns generated by the motion assembly 116.

With continued reference to FIG. 5 and FIG. 6, the rubber tires 190 provide ground contact and traction for the mechanical horse 100 during therapeutic sessions and mount directly to the rear axle 310 through hub assemblies that distribute the vehicle weight across the contact surfaces. The rubber tires 190 incorporate tread patterns and compound formulations that provide adequate traction on various surface conditions encountered during the circular therapy path operations. The rubber tires 190 support the combined weight of the mechanical horse 100 structure and individuals weighing over 300 pounds while maintaining stability throughout the therapy procedure that includes completing several laps, followed by a pause, then repeating this sequence with specific drills or exercises conducted during each pause. The rubber tires 190 coordinate with the steering assembly components, including the spindle 182 and the spindle linkage 186, to provide directional control during the counter-clockwise navigation around the therapy circle.

As further shown in FIG. 2, additional rubber tires 190 connect to the steering assembly through the spindle 182 and provide front-wheel steering capability for the mechanical horse 100. The front rubber tires 190 respond to directional commands from the direction switch 210 through the actuator 184, which drives the actuator arm 188 to position the spindle linkage 186 and orient the front rubber tires 190 for the circular therapy path. The front rubber tires 190 work in coordination with the rear rubber tires 190 to provide four-wheel mobility that supports the mechanical horse 100 during the complete treatment protocol. The rubber tires 190 accommodate the loading conditions created by the first rotatable platform section 104 and the second rotatable platform section 105 as these platform sections move in an arc of about 1 to 10 degrees, ensuring that the mobility system operates independently from the therapeutic motion patterns generated by the curved sheets 126, 128 that comprise fiberglass molds of a horse body.

Referring to FIG. 3, the drive system integrates with the motion assembly 116 through mechanical connections that coordinate the translational movement of the mechanical horse 100 with the therapeutic motion patterns generated by the cam-driven mechanism. The drive motor 300 operates under control of the speed rheostat 212, which regulates the rotational speed of the rear axle 310 to maintain the prescribed travel speeds during therapeutic operations. The drive chain 187 transfers power from the drive motor 300 through the transmission 197 to the chain sprocket 195, which connects to the rear axle 310 and drives the rear rubber tires 190. The drive system components coordinate with the first cam 192 and the second cam 292 to ensure that the translational movement of the mechanical horse 100 operates independently from the vertical displacement patterns generated by the left frame member 191 and the right frame member 291. The pillow block 198 provides rotational support for the cam shaft 193 while accommodating the mechanical loads generated by the coordinated operation of the drive system and the motion assembly 116.

The mobility components enable individuals who mount the mechanical horse 100 in a backward-facing position during therapy to experience consistent circular motion throughout the therapeutic protocol while the curved sheets 126, 128 generate the characteristic hip movement patterns through the coordinated operation of the rear legs 106, 107 and the front legs 125, 127. As shown in FIG. 7, the rubber tires 190 provide stable ground contact during the directional changes controlled by the direction switch 210, with the GPS system 209 integrated into the control panel 200 providing position tracking and safety monitoring during the mobility operations. The drive motor 300 receives electrical power from the 12-volt battery 320, which mounts to the battery support 322 attached to the top horizontal member 122, with the electrical disconnect 213 providing safety shutdown capability during therapeutic sessions. The drive system accommodates the structural loads generated when the mechanical horse 100 travels at speeds of up to 4 miles per hour while supporting individuals weighing over 300 pounds and maintaining the therapeutic motion patterns throughout the complete treatment protocol.

Referring to FIG. 4, the control panel 200 provides comprehensive operational control for the mechanical horse 100 through a centralized interface that integrates multiple control elements within the panel housing 202. The panel housing 202 encases the electrical components and control circuits that regulate the various operational parameters of the mechanical horse 100, including speed control, motion frequency adjustment, directional settings, and power management functions. The panel housing 202 incorporates protective construction that shields the internal control electronics from environmental conditions encountered during therapeutic operations while providing accessible mounting locations for the external control interfaces. The control panel 200 connects to the mechanical horse 100 through electrical pathways that route through the junction box 183, establishing communication links between the user interface elements and the operational systems including the drive motor 300, the motion assembly 116, and the steering components.

The speed rheostat 212 provides variable control over the translational velocity of the mechanical horse 100 and connects directly to the drive motor 300 through electrical circuits housed within the panel housing 202. The speed rheostat 212 incorporates rotational adjustment mechanisms that enable operators to select travel speeds ranging from stationary operation up to 4 miles per hour during therapeutic sessions. The electrical output from the speed rheostat 212 regulates the power delivered to the drive motor 300, which drives the rear axle 310 and the rubber tires 190 to propel the mechanical horse 100 around the circular therapy path. The speed rheostat 212 coordinates with the motion assembly 116 to ensure that the translational movement operates independently from the therapeutic motion patterns generated by the first cam 192 and the second cam 292. The speed control functionality enables the mechanical horse 100 to maintain consistent velocities while accommodating individuals weighing over 300 pounds who sit in a riding position or in a chair configuration during the therapy procedure that includes completing several laps, followed by a pause, then repeating this sequence with specific drills or exercises conducted during each pause.

With continued reference to FIG. 4, the motion rheostat 204 regulates the frequency of the vertical movement patterns generated by the motion assembly 116 and provides independent control over the therapeutic motion characteristics. The motion rheostat 204 connects to the cam shaft 193 through electrical circuits that control the rotational speed of the first cam 192 and the second cam 292, which drive the left frame member 191 and the right frame member 291 to create the alternating displacement patterns. The motion rheostat 204 enables operators to adjust the frequency of the vertical stroke of the curved sheets 126, 128 to optimize the therapeutic benefits delivered during each treatment session. The electrical output from the motion rheostat 204 coordinates with the transmission 197 to maintain the gear ratios that control the rotational speed of the cam shaft 193 while the mechanical horse 100 travels counter-clockwise around a 45-to 55-foot diameter circle. The motion control functionality operates independently from the speed rheostat 212, allowing simultaneous adjustment of both translational velocity and therapeutic motion frequency to accommodate the specific treatment protocols for individuals who mount the mechanical horse 100 in a backward-facing position during therapy.

The toggle switch 210 provides three-position directional control for the steering actuator 184 (FIG. 2) and incorporates self-centering functionality that returns the switch to a neutral position when released. The toggle switch 210 enables operators to select left, right, and neutral steering settings through mechanical switching actions that control the electrical polarity delivered to the steering actuator 184. The left position of the toggle switch 210 energizes the steering actuator 184 to turn the mechanical horse 100 in the counter-clockwise direction around the therapy circle, while the right position enables right turn movement for positioning adjustments. The neutral position of the toggle switch 210 disconnects power to the steering actuator 184 while maintaining electrical connections to the motion assembly 116, allowing the therapeutic motion patterns to continue operating while the mechanical horse 100 remains stationary. The self-centering design of the toggle switch 210 provides automatic return to the neutral position when operators release the switch, enhancing safety during therapeutic operations.

As further shown in FIG. 4, the toggle switch 211 controls the head light assembly component and provides operators with the ability to provide therapy in low light. The direction switch 210 connects to the actuator 184 through electrical circuits housed within the panel housing 202, enabling remote control of the actuator arm 188 and the spindle linkage 186 that position the front wheels for directional changes. The direction switch 210 coordinates with the spindle 182 to establish the steering angles needed for the mechanical horse 100 to maintain the prescribed circular therapy path while accommodating individuals weighing over 300 pounds. The electrical output from the direction switch 210 drives the actuator 184, which moves the actuator arm 188 to position the spindle linkage 186 and orient the front rubber tires 190 for the circular navigation pattern. The directional control functionality operates independently from the speed rheostat 212 and the motion rheostat 204, allowing operators to adjust steering settings while maintaining consistent translational velocity and therapeutic motion patterns throughout the therapy procedure.

The power switch 208 provides master electrical control for the mechanical horse 100 and connects to the 12-volt battery 320 through the primary power distribution circuits housed within the panel housing 202. The power switch 208 incorporates on/off switching functionality that controls electrical power delivery to all operational systems including the drive motor 300, the motion assembly 116, the steering components, and the GPS system 209. The power switch 208 coordinates with the battery support 322 that attaches to the top horizontal member 122 to provide stable mounting for the 12-volt battery 320 that powers the control panel 200 and the drive motor 300. The electrical connections from the power switch 208 route through the junction box 183 to distribute power to the various operational systems while maintaining circuit protection and safety features. The power switch 208 enables operators to energize the mechanical horse 100 for therapeutic sessions while providing immediate shutdown capability when individuals mount the mechanical horse 100 in a backward-facing position during therapy sessions that involve completing several laps around the circular path.

Referring to FIG. 5 and FIG. 7, the control panel 200 mounts to the mechanical horse 100 through a sophisticated positioning system that incorporates the vertical support 207 and the rotatable support 206 to provide adjustable placement of the user interface elements. The vertical support 207 extends upward from the rectangular horizontal frame 110 and provides the primary structural connection between the control panel 200 and the mechanical horse 100 framework. The vertical support 207 incorporates telescoping or fixed-height construction that positions the control panel 200 at accessible heights for operators while maintaining structural stability during therapeutic operations. The rotatable support 206 connects the control panel 200 to the vertical support 207 and enables angular positioning adjustments that optimize the orientation of the user interface elements relative to the operator position. The rotatable support 206 incorporates bearing assemblies or pivot mechanisms that allow the control panel 200 to rotate about the vertical axis established by the vertical support 207, providing flexibility in positioning the speed rheostat 212, the motion rheostat 204, the toggle switch 210, the direction switch 211, and the power switch 208 for optimal accessibility.

The mounting system formed by the vertical support 207 and the rotatable support 206 accommodates the operational requirements of therapeutic sessions where the mechanical horse 100 travels counter-clockwise around a circle at speeds of up to 4 miles per hour while generating therapeutic motion patterns through the first rotatable platform section 104 and the second rotatable platform section 105 that move in an arc of about 1 to 10 degrees. The vertical support 207 maintains structural integrity under the loading conditions created when individuals weighing over 300 pounds sit in a riding position or in a chair configuration on the curved sheets 126, 128 that comprise fiberglass molds of a horse body. The rotatable support 206 enables operators to adjust the control panel 200 orientation throughout the therapy procedure that includes completing several laps, followed by a pause, then repeating this sequence with specific drills or exercises conducted during each pause. The mounting system coordinates with the carrying handle 205 to provide portable positioning of the control panel 200 during setup and adjustment procedures for the typical individual sessions that comprise the complete therapeutic treatment protocol.

With continued reference to FIG. 4, the electrical disconnect 213 provides emergency safety functionality for the mechanical horse 100 and incorporates tethered design features that enable immediate power disconnection during therapeutic operations. The electrical disconnect 213 connects between the 12-volt battery 320 and the primary power distribution circuits within the panel housing 202, establishing a fail-safe mechanism that interrupts electrical power to all operational systems when activated. The tethered design of the electrical disconnect 213 incorporates a physical connection cord that attaches to the operator or a fixed reference point, automatically activating the disconnect function if the tether extends beyond a predetermined length. The electrical disconnect 213 coordinates with the power switch 208 to provide redundant safety features that protect individuals who mount the mechanical horse 100 in a backward-facing position during therapy sessions. The disconnect functionality interrupts power to the drive motor 300, the motion assembly 116, the steering components, and all control circuits while maintaining the structural integrity of the mechanical horse 100 framework including the rectangular horizontal frame 110, the curved sheets 126, 128, and the support elements.

As shown in FIG. 4, the GPS system 209 integrates into the control panel 200 and provides position tracking, progress logging, and safety monitoring capabilities during therapeutic operations. The GPS system 209 incorporates satellite positioning technology that tracks the location of the mechanical horse 100 throughout the therapy procedure and records movement patterns for analysis and documentation purposes. The GPS system 209 connects to the electrical circuits within the panel housing 202 and receives power from the 12-volt battery 320 through the power distribution system controlled by the power switch 208. The position tracking functionality of the GPS system 209 monitors the circular path navigation as the mechanical horse 100 travels counter-clockwise around the 45 to 55 foot diameter or similar circle, providing verification that the prescribed therapy protocol is followed correctly. The progress logging capabilities of the GPS system 209 record session data including travel distances, speeds, motion frequencies, and session durations for the typical ten or more individual sessions that comprise the complete therapeutic treatment protocol.

The safety features of the GPS system 209 include boundary monitoring that alerts operators if the mechanical horse 100 deviates from the prescribed therapy area, and emergency location broadcasting that transmits position information in the event of system malfunctions or safety incidents. The GPS system 209 coordinates with the electrical disconnect 213 to provide comprehensive safety monitoring during therapeutic sessions where individuals weighing over 300 pounds sit in a riding position or in a chair configuration while the first rotatable platform section 104 and the second rotatable platform section 105 generate therapeutic motion patterns through the coordinated operation of the first cam 192, the second cam 292, and the curved sheets 126, 128. The GPS system 209 operates independently from the speed rheostat 212 and the motion rheostat 204, providing continuous monitoring functionality regardless of the operational settings selected for translational velocity and therapeutic motion frequency during the therapy sessions that involve completing several laps, followed by a pause, then repeating this sequence with specific drills or exercises conducted during each pause.

Referring to FIG. 5, the battery 320 provides electrical power for all operational systems of the mechanical horse 100 and incorporates 12-volt specifications that deliver sufficient energy capacity for extended therapeutic sessions. The battery 320 connects to the drive motor 300 through electrical circuits that route through the junction box 183, establishing the primary power distribution pathway for the mobility system. The battery 320 supplies electrical energy to the control panel 200, enabling operation of the speed rheostat 212, the motion rheostat 204, the toggle switch 211, the direction switch 210, and the power switch 208 throughout therapeutic sessions where individuals weighing over 300 pounds sit in a riding position or in a chair on the mechanical horse 100. The electrical output from the battery 320 maintains consistent voltage levels during the therapy procedure that includes completing several laps around a 45-to 55-foot diameter circle, followed by a pause, then repeating this sequence with specific drills or exercises conducted during each pause.

The battery support 322 secures the battery 320 to the top horizontal member 122 and provides stable mounting that distributes the weight of the battery 320 across the structural framework of the rectangular horizontal frame 110. The battery support 322 incorporates mounting brackets and retention mechanisms that maintain the position of the battery 320 during the operational cycles when the first rotatable platform section 104 and the second rotatable platform section 105 move in an arc of about 1 to 10 degrees. The attachment of the battery support 322 to the top horizontal member 122 positions the battery 320 at an elevated location within the mechanical horse 100 structure, providing protection from ground contact while maintaining accessibility for maintenance procedures. The battery support 322 accommodates the loading conditions created when the mechanical horse 100 travels counter-clockwise around the circular therapy path while supporting individuals who mount the mechanical horse 100 in a backward-facing position during therapy.

With continued reference to FIG. 5, the electrical connections between the battery 320 and the operational systems route through protective pathways that integrate with the rectangular horizontal frame 110 and connect to the junction box 183 for distribution to the various electrical components. The battery 320 powers the motion assembly 116 through electrical circuits that control the rotational speed of the cam shaft 193, enabling the first cam 192 and the second cam 292 to generate the therapeutic motion patterns through the left frame member 191 and the right frame member 291. The electrical energy from the battery 320 drives the actuator 184 within the steering assembly, providing power for the actuator arm 188 to position the spindle linkage 186 about the spindle 182 for directional control during therapeutic operations. The battery 320 maintains electrical power delivery throughout the complete treatment protocol that typically requires ten or more individual sessions, with the 12-volt specifications providing adequate energy capacity for the extended operational periods associated with therapeutic applications.

As shown in FIG. 7, the battery 320 integrates with the overall electrical architecture of the mechanical horse 100 through connections that coordinate with the GPS system 209 and the electrical disconnect 213 to provide comprehensive power management and safety features. A battery charger connects to the battery 320 through electrical interfaces that enable recharging between therapeutic sessions, with the battery charger provided as a separate unit or integrated into the control panel 200 depending on the specific configuration requirements. The battery charger incorporates charging circuits that restore the electrical capacity of the battery 320 while maintaining the voltage specifications needed for the drive motor 300, the control panel 200, and the motion assembly 116. The charging system coordinates with the battery support 322 to provide accessible connections for the battery charger while maintaining the secure mounting of the battery 320 to the top horizontal member 122 during charging operations.

Referring to FIG. 4, the GPS system 209 integrates into the control panel 200 and provides advanced positioning and monitoring capabilities that enhance the safety and effectiveness of therapeutic operations. The GPS system 209 incorporates satellite positioning technology that tracks the location of the mechanical horse 100 throughout the therapy sessions and records movement patterns for analysis and documentation purposes. The GPS system 209 receives electrical power from the battery 320 through the power distribution circuits housed within the panel housing 202, with the power switch 208 controlling the electrical energy delivery to the GPS system 209 components. The positioning data from the GPS system 209 monitors the circular path navigation as the mechanical horse 100 travels counter-clockwise around the prescribed therapy area, providing verification that the treatment protocol follows the correct movement patterns for individuals who sit in a riding position or in a chair configuration.

The GPS system 209 incorporates progress logging functionality that records session data including travel distances, operational speeds, motion frequencies, and session durations for the therapeutic treatments. The data logging capabilities of the GPS system 209 track the movement patterns generated by the first rotatable platform section 104 and the second rotatable platform section 105 as these platform sections move in an arc of about 1 to 10 degrees during therapeutic operations. The GPS system 209 coordinates with the curved sheets 126, 128 that comprise fiberglass molds of a horse body to provide comprehensive monitoring of the therapeutic motion patterns delivered during each session. The recorded data from the GPS system 209 supports analysis of the treatment effectiveness and provides documentation for the sessions that comprise the complete therapeutic protocol.

With continued reference to FIG. 4, the GPS system 209 incorporates safety features that include boundary monitoring and emergency location broadcasting capabilities that protect individuals who mount the mechanical horse 100 in a backward-facing position during therapy. The boundary monitoring function of the GPS system 209 alerts operators if the mechanical horse 100 deviates from the prescribed therapy area during the procedure that includes completing several laps, followed by a pause, then repeating this sequence with specific drills or exercises conducted during each pause. The emergency location broadcasting capability of the GPS system 209 transmits position information in the event of system malfunctions or safety incidents, coordinating with the electrical disconnect 213 to provide comprehensive safety monitoring during therapeutic sessions. The GPS system 209 operates independently from the speed rheostat 212 and the motion rheostat 204, providing continuous monitoring functionality regardless of the operational settings selected for translational velocity and therapeutic motion frequency.

The mechanical horse 100 may include a GPS device designed specifically for tracking the location of the mechanical horse 100. The GPS device may include various components that work together to accurately determine and monitor the position of the mechanical horse 100. The GPS device may be attached to the mechanical horse, enabling real-time tracking of the mechanical horse location. The GPS device may utilize satellite signals to determine the precise location of the mechanical horse and may relay this information to a user through a variety of means, such as a mobile application or a web-based platform. In some cases, the GPS device may also include features that allow for historical tracking data to be stored and accessed, providing a record of the mechanical horse movements over time. The GPS device may include an alarm feature that can be activated when the mechanical horse is moved beyond a predetermined distance from the user's device. This feature may help prevent the loss of the mechanical horse by alerting the user when the mechanical horse is moved out of range. In other cases, the GPS device may include a temperature sensor, providing information about the environment in which the mechanical horse is located. The GPS device may function by receiving signals from a network of satellites orbiting the Earth. These signals may be processed by the GPS device to determine the precise location of the mechanical horse. The location data may be calculated based on the time it takes for the signals to travel from the satellites to the GPS device. In some cases, the GPS device may use a method known as trilateration, which involves determining the location of the mechanical horse based on the intersection of spheres centered at the satellites.

The GPS device may include a processor for processing the satellite signals and calculating the location of the mechanical horse. This processor may be a microprocessor or a digital signal processor, among others. The processor may be configured to execute algorithms or software instructions stored in a memory module of the GPS device, which may facilitate the processing of the satellite signals and the calculation of the location data. The GPS device may include a receiver for receiving the satellite signals. This receiver may be designed to operate on specific frequencies used by the GPS satellites. The receiver may be capable of receiving signals from multiple satellites simultaneously, which may enhance the accuracy of the location data. The GPS device may transmit the location data to a user's device through a communication module. This communication module may utilize various communication protocols, such as Bluetooth, Wi-Fi, or cellular networks, to transmit the data. The user's device may display the location of the mechanical horse on a map, providing a visual representation of the mechanical horse location. In some cases, the user's device may also provide directions to the mechanical horse location, assisting the user in locating the mechanical horse. The GPS device may utilize various communication protocols to transmit the location data to the mobile application or the web-based platform. These communication protocols may include Bluetooth, Wi-Fi, or cellular networks, among others. The choice of communication protocol may depend on various factors, such as the distance between the GPS device and the user's device, the availability of network coverage, or the power consumption of the GPS device. In some cases, the GPS device may be capable of switching between different communication protocols based on these factors, ensuring reliable transmission of the location data.

As shown in FIG. 1 and FIG. 3, the junction box 183 provides centralized electrical connection and distribution functionality for the mechanical horse 100 and houses the electrical interfaces that connect the battery 320 to the various operational systems. The junction box 183 incorporates protective enclosure construction that shields the electrical connections from environmental conditions encountered during therapeutic operations while providing accessible connection points for maintenance and troubleshooting procedures. The junction box 183 connects to the drive motor 300 through electrical pathways that deliver power from the battery 320 to the mobility system, enabling the mechanical horse 100 to travel at speeds of up to 4 miles per hour while accommodating individuals weighing over 300 pounds. The electrical distribution function of the junction box 183 coordinates with the control panel 200 to provide power delivery to the speed rheostat 212, the motion rheostat 204, the toggle switch 211, the direction switch 210, and the power switch 208.

The junction box 183 incorporates circuit protection features that safeguard the electrical systems from overcurrent conditions and voltage fluctuations that could occur during therapeutic operations. The protective circuits within the junction box 183 coordinate with the electrical disconnect 213 to provide redundant safety features that protect the battery 320, the drive motor 300, and the control panel 200 from electrical faults. The junction box 183 houses the electrical connections that power the motion assembly 116, including the circuits that control the rotational speed of the cam shaft 193 and the operation of the first cam 192 and the second cam 292. The electrical pathways within the junction box 183 distribute power to the actuator 184 within the steering assembly, enabling the actuator arm 188 to position the spindle linkage 186 for directional control during the circular therapy path navigation.

Referring to FIG. 2, the junction box 183 coordinates with the steering assembly components to provide electrical power for the actuator 184 that drives the directional control functions during therapeutic operations. The electrical connections from the junction box 183 enable the actuator 184 to respond to commands from the direction switch 210, positioning the actuator arm 188 to move the spindle linkage 186 about the spindle 182 for steering control. The junction box 183 distributes electrical power to the rubber tires 190 through the drive system connections, coordinating with the rear axle 310 to provide mobility for the mechanical horse 100 during the therapy sessions. The electrical architecture established by the junction box 183 supports the coordinated operation of the translational movement and the therapeutic motion patterns generated by the curved sheets 126, 128 through the body hinge 170 connections to the top horizontal member 122.

As further shown in FIG. 6, the junction box 183 integrates with the overall structural framework of the mechanical horse 100 through mounting connections to the rectangular horizontal frame 110 that provide stable positioning of the electrical distribution components. The junction box 183 coordinates with the non-slip pad 303 installations on the first rotatable platform section 104 and the second rotatable platform section 105 by providing electrical power for any integrated safety systems or monitoring devices associated with the platform sections. The electrical distribution function of the junction box 183 supports the complete therapeutic protocol by maintaining consistent power delivery throughout the treatment sessions, enabling the mechanical horse 100 to accommodate the loading conditions created when individuals mount the device in a backward-facing position and experience the therapeutic motion patterns generated by the coordinated operation of the motion assembly 116, the curved sheets 126, 128, and the split platform system.

Referring to FIG. 6 and FIG. 7, the non-slip pad 303 attaches to each of the first rotatable platform section 104 and the second rotatable platform section 105 to provide secure footing during mounting and dismounting procedures. The non-slip pad 303 incorporates surface textures and material compositions that enhance traction between user footwear and the platform surfaces, reducing the risk of slipping when individuals weighing over 300 pounds mount the mechanical horse 100 in a backward-facing position during therapy. The material construction of the non-slip pad 303 withstands the repetitive loading cycles associated with the therapy procedure that includes completing several laps, followed by a pause, then repeating this sequence with specific drills or exercises conducted during each pause. The non-slip pad 303 covers strategic areas of the first rotatable platform section 104 and the second rotatable platform section 105 where users place their feet during mounting procedures and while sitting in a riding position or in a chair on the mechanical horse 100. The surface pattern of the non-slip pad 303 incorporates raised textures or grooved configurations that channel moisture away from the contact areas while maintaining grip characteristics under various environmental conditions encountered during therapeutic sessions. The non-slip pad 303 coordinates with the curved sheets 126, 128 that comprise fiberglass molds of a horse body to provide comprehensive safety features throughout the mounting and riding experience. The attachment method for the non-slip pad 303 utilizes adhesive bonding or mechanical fastening techniques that maintain secure positioning on the platform sections during the operational cycles.

With continued reference to FIG. 6, the positioning of the non-slip pad 303 on the first rotatable platform section 104 and the second rotatable platform section 105 accommodates the foot placement patterns of individuals who mount the mechanical horse 100 in a backward-facing position during therapy sessions. The non-slip pad 303 extends across sufficient surface area to provide stable footing during the dynamic conditions created when the platform sections articulate through their rotational range while supporting the weight of users. The thickness specifications of the non-slip pad 303 provide cushioning effects that reduce pressure concentrations on user feet while maintaining the structural integrity of the underlying platform sections. The non-slip pad 303 integrates with the rectangular horizontal frame 110 through the platform mounting system formed by the first hinge 102 and the second hinge 103, ensuring that the safety features remain effective throughout the complete treatment protocol.

FIG. 6 and FIG. 7 show the first rotatable platform section 104 rotatable in an arc 402 whereby an outer edge of the first rotatable platform section 104 moves primarily in a vertical direction with respect to the frame and an inner edge of the first rotatable platform section 104 is vertically stationary with respect to the frame and only rotates about the hinge to which the inner edge is attached. The second rotatable platform section 105 rotatable in an arc 400 whereby an outer edge of the second rotatable platform section 105 moves primarily in a vertical direction with respect to the frame and an inner edge of the second rotatable platform section 105 is vertically stationary with respect to the frame and only rotates about the hinge to which the inner edge is attached. The first rotatable platform section 104 is shown in an angular position different than the angular position of the second rotatable platform section 105, whereby legs 107, 127 are shown having rotated the curved sheet 128, positioning the second rotatable platform section 105 higher than first rotatable platform section 104. The movement of the first rotatable platform section 104 and the second rotatable platform section 105 in opposing stroke patterns causes the curved sheets 126, 128 to rotate in corresponding stroke patterns through the rear legs 106, 107 and the front legs 125, 127 which include a pivotal linkage at each leg end. The foam padding 130 and the saddle pad 140 provide additional comfort to the rider. The stroke patterns allow movement of the curved sheets to mimic the movement of hips of a horse.

As shown in FIG. 4, the electrical disconnect 213 provides emergency safety functionality that enables immediate power disconnection during therapeutic operations through tethered design features that activate automatically when safety conditions require intervention. The electrical disconnect 213 connects between the battery 320 and the primary power distribution circuits within the panel housing 202, establishing a fail-safe mechanism that interrupts electrical power to all operational systems including the drive motor 300, the motion assembly 116, and the steering components. The tethered design of the electrical disconnect 213 incorporates a physical connection cord that attaches to the operator or a fixed reference point, automatically activating the disconnect function if the tether extends beyond a predetermined length during therapeutic sessions. The electrical disconnect 213 coordinates with the power switch 208 to provide redundant safety features that protect individuals who sit in a riding position or in a chair configuration while the curved sheets 126, 128 generate therapeutic motion patterns through the coordinated operation of the first cam 192 and the second cam 292.

Referring to FIG. 1 and FIG. 5, the body hinge 170 incorporates safety features that limit the range of motion for the curved sheets 126, 128 to prevent excessive displacement that could compromise user stability during therapeutic operations. The body hinge 170 establishes mechanical stops or limiting mechanisms that constrain the articulation of the curved sheets 126, 128 within safe operational parameters while the first rotatable platform section 104 and the second rotatable platform section 105 move in an arc of about 1 to 10 degrees. The safety limiting function of the body hinge 170 coordinates with the foam padding 130 and the saddle pad 140 to provide comprehensive protection for individuals weighing over 300 pounds during the therapy procedure that includes completing several laps, followed by a pause, then repeating this sequence with specific drills or exercises conducted during each pause. The body hinge 170 incorporates bearing assemblies or pivot mechanisms that maintain smooth operation while providing the mechanical constraints needed to prevent unsafe motion conditions.

As further shown in FIG. 4, the GPS system 209 integrates safety monitoring capabilities that track the position of the mechanical horse 100 and provide boundary monitoring functions that alert operators if the device deviates from the prescribed therapy area. The GPS system 209 incorporates satellite positioning technology that monitors the circular path navigation as the mechanical horse 100 travels counter-clockwise around a 45-to 55-foot diameter circle, providing verification that the treatment protocol follows the correct movement patterns for therapeutic effectiveness. The boundary monitoring function of the GPS system 209 coordinates with the electrical disconnect 213 to provide automatic safety responses when the mechanical horse 100 approaches predetermined safety boundaries during therapeutic sessions. The emergency location broadcasting capability of the GPS system 209 transmits position information in the event of system malfunctions or safety incidents, enabling rapid response from support personnel during therapeutic operations where individuals mount the mechanical horse 100 in a backward-facing position.

With reference to FIG. 2, the actuator mount 180 incorporates safety features that secure the actuator 184 to prevent displacement during steering operations that could compromise directional control of the mechanical horse 100. The actuator mount 180 distributes the mechanical loads generated during directional changes across the rectangular horizontal frame 110, ensuring structural integrity when the mechanical horse 100 accommodates individuals weighing over 300 pounds who sit in a riding position or in a chair configuration. The mounting design of the actuator mount 180 incorporates redundant fastening mechanisms that maintain the position of the actuator 184 throughout the operational cycles associated with the circular therapy path navigation. The actuator mount 180 coordinates with the spindle 182 and the spindle linkage 186 to provide stable steering control while the rubber tire 190 responds to directional commands from the direction switch 210 during therapeutic sessions.

Referring to FIG. 3, the pillow block 198 provides rotational support for the cam shaft 193 while incorporating safety features that prevent mechanical failure during the repetitive loading cycles generated by the motion assembly 116. The pillow block 198 incorporates bearing assemblies that accommodate the rotational loads created when the first cam 192 and the second cam 292 drive the left frame member 191 and the right frame member 291 to generate therapeutic motion patterns. The structural design of the pillow block 198 distributes the mechanical stresses across the rectangular horizontal frame 110, preventing localized failure points that could compromise the safety of the motion assembly 116 during therapeutic operations. The pillow block 198 coordinates with the drive chain 187 and the transmission 197 to maintain precise alignment of the cam shaft 193 while the mechanical horse 100 travels and generates the therapeutic motion patterns through the curved sheets 126, 128 that comprise fiberglass molds of a horse body.

As shown in FIG. 5, the battery support 322 attaches to the top horizontal member 122 and incorporates safety features that secure the battery 320 to prevent displacement during therapeutic operations that could create electrical hazards or compromise power delivery to the operational systems. The battery support 322 distributes the weight of the battery 320 across the structural framework of the rectangular horizontal frame 110, preventing localized stress concentrations that could compromise structural integrity when the mechanical horse 100 accommodates individuals weighing over 300 pounds. The mounting design of the battery support 322 incorporates retention mechanisms that maintain the position of the battery 320 during the operational cycles when the first rotatable platform section 104 and the second rotatable platform section 105 move through their rotational range. The battery support 322 coordinates with the junction box 183 to provide protected electrical connections that route power from the battery 320 to the drive motor 300, the control panel 200, and the motion assembly 116 while maintaining safety isolation from user contact areas during therapeutic sessions where individuals mount the mechanical horse 100 in a backward-facing position and experience the complete treatment protocol.

Referring to FIG. 1, the therapeutic operation of the mechanical horse 100 begins with individuals mounting the device in a backward-facing position, which establishes the standard positioning protocol for the treatment sessions. The backward-facing mounting position places users with their back toward the direction of travel while the mechanical horse 100 travels counter-clockwise. The curved sheets 126, 128 that comprise fiberglass molds of a horse body provide anatomically accurate seating surfaces that accommodate individuals weighing over 300 pounds who sit in a riding position or in a chair configuration during therapeutic operations. The foam padding 130 and the saddle pad 140 enhance comfort during the extended treatment sessions while the body hinge 170 enables the therapeutic rocking motion that characterizes the treatment protocol. The backward-facing orientation coordinates with the circular movement patterns to create specific neurological stimulation effects that promote therapeutic benefits through the coordinated operation of the first rotatable platform section 104 and the second rotatable platform section 105.

With continued reference to FIG. 1, the circular movement patterns generated during therapeutic operations incorporate precise geometric specifications that establish the spatial parameters for the treatment protocol. The mechanical horse 100 follows a counter-clockwise path around a circle with a diameter ranging from 45 to 55 feet, creating consistent movement patterns that coordinate with the therapeutic motion generated by the motion assembly 116. The first rotatable platform section 104 and the second rotatable platform section 105 move in an arc of about 1 to 10 degrees during the circular navigation, creating alternating displacement patterns that transfer through the bottom linkage 108 to the rear legs 106, 107 and the front legs 125, 127. The curved sheets 126, 128 respond to the platform motion through the body hinge 170 connections to the top horizontal member 122, generating the characteristic hip movement patterns that simulate equine gait characteristics. The circular path dimensions coordinate with the speed specifications to create specific temporal patterns that enhance the therapeutic effectiveness of the treatment sessions.

As shown in FIG. 4, the control panel 200 regulates the operational parameters of the therapeutic protocol through the coordinated operation of the speed rheostat 212 and the motion rheostat 204. The speed rheostat 212 controls the translational velocity of the mechanical horse 100 as the device travels around the circular therapy path, maintaining speeds up to 4 miles per hour throughout the treatment sessions. The motion rheostat 204 regulates the frequency of the vertical movement patterns generated by the motion assembly 116, controlling the therapeutic motion characteristics delivered through the curved sheets 126, 128. The toggle switch 210 enables operators to select left, right, and neutral steering settings during the treatment protocol, while the direction switch 211 controls the steering angles needed to maintain the counter-clockwise circular path. The power switch 208 provides master control for the therapeutic operations, coordinating with the 12-volt battery 320 to deliver electrical power to the drive motor 300 and the control systems throughout the treatment sessions.

Referring to FIG. 3, the motion assembly 116 generates the therapeutic movement patterns through the coordinated operation of the first cam 192 and the second cam 292, which drive the left frame member 191 and the right frame member 291 to create alternating vertical displacement patterns. The cam shaft 193 rotates within the pillow block 198 to drive the cam mechanisms, with the drive chain 187 transferring power from the transmission 197 to maintain the rotational speeds that generate the therapeutic motion frequencies. The first rotatable platform section 104 and the second rotatable platform section 105 respond to the cam-driven motion by moving in an arc of about 1 to 10 degrees, creating the angular displacement patterns that transfer through the mechanical linkages to the curved sheets 126, 128. The vertical movement indicator 199 illustrates the range of motion generated by the cam system, with the alternating displacement patterns creating the characteristic hip movement simulation that provides therapeutic benefits during the treatment protocol. The motion assembly 116 operates independently from the translational movement of the mechanical horse 100, enabling simultaneous generation of circular path navigation and therapeutic motion patterns throughout the treatment sessions.

With reference to FIG. 5, the treatment protocol incorporates a structured sequence of operational phases that include completing several laps around the circular path, followed by a pause, then repeating this sequence with specific drills or exercises conducted during each pause. The lap phase involves continuous circular movement where the mechanical horse 100 travels counter-clockwise around the 45-to 55-foot diameter circle while the curved sheets 126, 128 generate therapeutic motion patterns through the coordinated operation of the first rotatable platform section 104 and the second rotatable platform section 105. The drive motor 300 provides consistent propulsion through the rear axle 310 and the rubber tires 190, maintaining the prescribed travel speeds while individuals weighing over 300 pounds experience the therapeutic motion patterns in the backward-facing mounting position. The pause phase interrupts the circular movement while maintaining the therapeutic motion generated by the motion assembly 116, allowing operators to conduct specific drills or exercises that enhance the treatment effectiveness. The battery 320 provides electrical power throughout the complete lap-pause-drill sequence, with the battery support 322 attached to the top horizontal member 122 maintaining stable power delivery during the operational cycles.

As further shown in FIG. 4, the GPS system 209 integrated into the control panel 200 tracks the position of the mechanical horse 100 throughout the lap-pause-drill methodology and logs progress data for analysis and documentation purposes. The GPS system 209 monitors the circular path navigation during the lap phases, recording movement patterns and verifying that the mechanical horse 100 maintains the prescribed counter-clockwise direction around the therapy circle. The position tracking functionality coordinates with the pause phases by documenting the locations where specific drills or exercises occur, creating comprehensive session records that support treatment analysis. The progress logging capabilities of the GPS system 209 record operational parameters including travel distances, speeds, motion frequencies, and session durations for each lap-pause-drill cycle. The safety features of the GPS system 209 provide boundary monitoring that alerts operators if the mechanical horse 100 deviates from the prescribed therapy area during any phase of the treatment protocol, coordinating with the electrical disconnect 213 to provide comprehensive safety oversight throughout the therapeutic operations.

Referring to FIG. 6, the complete treatment protocol incorporates a plurality of sessions that follow the lap-pause-drill methodology to achieve the therapeutic objectives for individuals who mount the mechanical horse 100 in the backward-facing position. Each individual session includes multiple cycles of the lap-pause-drill sequence, with the specific number of laps and the duration of pause periods adjusted according to the treatment requirements for each individual. The non-slip pad 303 on the first rotatable platform section 104 and the second rotatable platform section 105 provides secure footing during mounting and dismounting procedures at the beginning and end of each individual session. The saddle pad 140 maintains comfort throughout the extended treatment sessions while the curved sheets 126, 128 deliver consistent therapeutic motion patterns through the coordinated operation of the motion assembly 116. The control panel 200 enables operators to adjust the operational parameters between individual sessions, optimizing the speed settings through the speed rheostat 212 and the motion characteristics through the motion rheostat 204 to accommodate the progressive treatment requirements across the session protocols.

With continued reference to FIG. 6, a plurality of individual sessions incorporate progressive adjustments to the operational parameters that enhance the therapeutic effectiveness as individuals adapt to the treatment protocol. The initial sessions focus on establishing comfort with the backward-facing mounting position and the circular movement patterns, with lower speeds and reduced motion frequencies to facilitate adaptation to the therapeutic environment. The intermediate sessions increase the operational parameters as individuals become accustomed to the lap-pause-drill methodology, incorporating more complex drills and exercises during the pause phases to enhance the therapeutic benefits. The final sessions utilize the full operational capabilities of the mechanical horse 100, with speeds up to 4 miles per hour and maximum motion frequencies generated by the first rotatable platform section 104 and the second rotatable platform section 105 moving through their complete arc of about 1 to 10 degrees. The rear axle 310 and the rubber tires 190 provide consistent mobility throughout the sessions, with the drive motor 300 maintaining reliable propulsion while accommodating individuals weighing over 300 pounds throughout the complete treatment protocol.

As shown in FIG. 7, the therapeutic operation coordinates multiple operational systems to deliver the complete treatment experience through the integration of mobility, motion generation, and control functions. The mechanical horse 100 accommodates individuals who sit in a riding position or in a chair configuration while maintaining the backward-facing orientation throughout the lap phases of each treatment session. The junction box 183 distributes electrical power from the 12-volt battery 320 to the operational systems, enabling coordinated operation of the drive motor 300, the motion assembly 116, and the control panel 200 throughout the individual sessions. The direction switch 212 and the toggle switch 211 provide operational control during the lap-pause-drill sequences, with the GPS system 209 monitoring the treatment progress and logging session data for analysis. The non-slip pad 303 enhances safety during the mounting and dismounting procedures that occur at the beginning and end of each individual session, while the curved sheets 126, 128 that comprise fiberglass molds of a horse body provide anatomically accurate therapeutic surfaces throughout the complete treatment protocol.

Referring to FIG. 2, the steering assembly coordinates with the therapeutic operation by maintaining precise directional control during the circular movement patterns that characterize the lap phases of each treatment session. The spindle 182 and the spindle linkage 186 respond to commands from the direction switch 210 through the actuator 184 and the actuator arm 188, positioning the rubber tires 190 to maintain the counter-clockwise path around the 45-to 55-foot diameter circle. The actuator mount 180 provides stable support for the steering components while the mechanical horse 100 accommodates individuals weighing over 300 pounds during the therapeutic operations. The steering assembly operates independently from the therapeutic motion patterns generated by the motion assembly 116, enabling simultaneous navigation control and therapeutic motion delivery throughout the lap-pause-drill methodology. The coordinated operation of the steering assembly with the drive motor 300 and the motion assembly 116 creates the complete therapeutic experience that spans the individual sessions of the treatment protocol, with each session incorporating multiple cycles of the lap-pause-drill sequence to achieve the therapeutic objectives for individuals who mount the mechanical horse 100 in the backward-facing position during therapy.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A mobile therapeutic mechanical horse for horseback sensory therapy, comprising:
a rectangular horizontal frame having a length and a central axis along the length;
a split platform having a first rotatable platform section and a second rotatable platform section, the first rotatable platform section having a first hinge attached along the central axis and the second rotatable platform section having a second hinge attached along the central axis, whereby the first rotatable platform section and the second rotatable platform section move in an arc;
a mechanical horse body for sitting of a person, the mechanical horse body including a front vertical support tube, a rear vertical support tube, a top horizontal member secured to upper portions of the front vertical support tube and the rear vertical support tube parallel to the frame length, two curved sheets each having a body hinge attached to the top horizontal member, two rear legs and two front legs each hinged on a top end to one of the two curved sheets, wherein each of the two rear legs and two front legs have a bottom linkage pivotally secured to the first rotatable platform section or the second rotatable platform section;
a motion assembly including a first cam for vertical movement of a left frame member wherein the left frame member is attached to a bottom surface of the first rotatable platform section and the first cam is attached to a cam shaft, and a second cam for vertical movement of a right frame member wherein the right frame member is attached to a bottom surface of the second rotatable platform section and the second cam is attached to the cam shaft;
a drive motor for driving wheels; and
a control panel including a speed rheostat for controlling speed of the mobile therapeutic mechanical horse and a motion rheostat for controlling frequency of vertical stroke of the curved sheets.

2. The mobile therapeutic mechanical horse of claim 1, further comprising a non-slip pad on each of the first rotatable platform section and the second rotatable platform section.

3. The mobile therapeutic mechanical horse of claim 1, wherein the two curved sheets comprise fiberglass molds of a horse body.

4. The mobile therapeutic mechanical horse of claim 3, further comprising foam padding disposed on each of the curved sheets and a saddle pad secured to the foam padding.

5. The mobile therapeutic mechanical horse of claim 1, further comprising a steering assembly including a spindle, a spindle linkage, an actuator arm for moving the spindle linkage about the spindle, and an actuator controlled by a direction switch.

6. The mobile therapeutic mechanical horse of claim 5, wherein the control panel further includes the direction switch for setting front wheels in a straight, right or left direction.

7. The mobile therapeutic mechanical horse of claim 1, wherein the cam shaft is rotatably secured to the rectangular horizontal frame by pillow blocks.

8. The mobile therapeutic mechanical horse of claim 7, further comprising a drive chain mechanically linking a chain sprocket with the cam shaft for driving the cam shaft.

9. The mobile therapeutic mechanical horse of claim 1, further comprising a battery secured by a battery support attached to the top horizontal member, wherein the battery is electrically connected to the control panel.

10. The mobile therapeutic mechanical horse of claim 9, wherein the control panel further includes a power switch, a three-position toggle switch for forward, reverse and neutral drive settings, and a tethered electrical disconnect.

11. A mobile therapeutic mechanical horse, comprising:
a frame;
a split platform having a first platform section and a second platform section that rotate independently about a central axis to create alternating motion patterns;
curved riding surfaces connected to the platform sections through mechanical linkages that transfer platform motion to simulate equine hip movement;
a cam-driven motion assembly that generates vertical displacement patterns to drive the platform sections;
a mobility system including a drive motor and wheels that enables the mechanical horse to travel in a circular path; and
a control system that independently regulates translational speed and therapeutic motion frequency.

12. The mobile therapeutic mechanical horse of claim 11, wherein the curved riding surfaces comprise fiberglass molds of a horse body that provide anatomically accurate contours for therapeutic positioning.

13. The mobile therapeutic mechanical horse of claim 12, further comprising foam padding disposed on each of the curved riding surfaces and a saddle pad secured to the foam padding.

14. The mobile therapeutic mechanical horse of claim 11, wherein the cam-driven motion assembly includes a first cam and a second cam mounted to a cam shaft at offset positions to create alternating vertical displacement patterns.

15. The mobile therapeutic mechanical horse of claim 14, further comprising pillow blocks that rotatably support the cam shaft within the frame and a drive chain that mechanically links the cam shaft to the drive motor.

16. A method of providing horseback sensory therapy using a mobile therapeutic mechanical horse, comprising:
positioning an individual on curved sheets of the mechanical horse in a backward-facing position;
operating a motion assembly to move a first rotatable platform section and a second rotatable platform section in alternating arcs degrees to create therapeutic hip movement patterns through the curved sheets;
propelling the mechanical horse in a counter-clockwise direction around a circular path;
completing a sequence of multiple laps followed by a pause; and
repeating the sequence with specific exercises conducted during each pause.

17. The method of claim 16, wherein the therapeutic hip movement patterns are generated by a cam-driven motion assembly that includes a first cam and a second cam mounted to a cam shaft at offset positions.

18. The method of claim 17, wherein the cam shaft is driven by a drive motor through a transmission and drive chain system that maintains gear ratios for proper motion speed limits.

19. The method of claim 16, further comprising a step of controlling the speed of the mechanical horse using a speed rheostat and controlling the frequency of the therapeutic hip movement patterns using a motion rheostat.

20. The method of claim 16, wherein the complete therapeutic treatment protocol comprises a plurality of individual sessions, each session including multiple sequences of the laps, pauses, and exercises.
